(19) United States
(12) Patent Application Publication
(10) Pub. No.: US 2006/0173359 A1
(43) Pub. Date: Aug. 3, 2006

(54) Optical apparatus for guided liver tumor treatment and methods

(76) Inventors: Wei Chiang Lin, Miami, FL (US); William C Chapman, St. Louis, MO (US); Anita Mahadevan-Jansen, Nashville, TN (US)

Correspondence Address: MORRIS MANNING & MARTIN LLP, 1600 ATLANTA FINANCIAL CENTER, 3343 PEACHTREE ROAD, NE, ATLANTA, GA, 30326-1044, US

(21) Appl. No.: 10/528,241

(22) PCT Filed: Sep. 30, 2003
(86) PCT No.: PCT/US03/31163

§ 371 Date: Dec. 2, 2005

Related U.S. Application Data
(60) Provisional application No. 60/415,282, filed on Sep. 30, 2002.

(51) Int. Cl. A61B 6/00

(52) U.S. Cl. 600/478

(57) ABSTRACT

Native tumorous and non-tumorous tissues and thermally denatured tissues can all be identified optically in patient livers by means of one or more probes inserted into the liver. Fluorescence and/or diffuse reflectance values can be used for initially locating a tumorous mass, effectively centering a thermal ablation device within a tumor, monitoring ablation of the tumor about the tumor to assure effective denaturation of the tumor, and locating the mass of denatured tissue after the thermal denaturation treatment and after the treated tissue has returned to the temperature of the untreated surrounding tissue. An existing thermal ablation probe can be modified by the provision of optical fiber within a hollow electrode to form an optical probe performing any of the above functions with an illuminating light source, a light detector and a processor.

### BACKGROUND OF THE INVENTION

Liver cancers pose a significant problem to public health worldwide, especially in some Asian and African countries. Over half a million new cases of primary liver cancers are reported globally every year. In addition, liver metasases are present in roughly half of all cancers. To make the situation worse, liver cancers are also associated with a very low, long-term survival rate. The five-year survival rate of patients with primary liver cancers in the United States, for example, is less than 10%. Surgical resection is currently the primary treatment option for liver cancers. However, the majority of liver cancer patients can not undergo surgical resection because of disease extent or location and their medical condition.

Therapies such as radio frequency thermal coagulation and laser-induced thermal coagulation are often considered as alternative treatments when resection of liver tumors is not a viable option. Currently, ablation probe placement is approximately determined in accordance with palpation and ‘free-hand’ ultrasound imaging. The accuracy of this approach, unfortunately, is hindered by the limitations of tumor margins detection using tumor echogenicity and stiffness. Currently, all thermotherapy procedures also suffer from the lack of an adequate feedback control system, making it difficult to know precisely when to cease coagulation. In current practice, clinicians rely on predetermined power and therapeutic duration (i.e., heating time) to conduct thermotherapies for liver tumors. This practice often yields unsatisfying therapeutic outcomes due to the fact that tissue characteristics vary drastically from patient to patient In addition, the dynamics of tissue characteristics, such as temperature-dependent tissue optics, further complicate the process of thermal coagulation of liver tissues. Since the coagulation zones are often not visible during thermotherapy, it is difficult, if not impossible, to precisely determine the end point of therapy. To avoid the undesired consequences resulting from under- or over-treatment (such as tumor recurrence), an effective feedback control strategy that provides an objective end point for thermotherapies of liver tumors is clearly needed.

Thermal coagulation of tissues (sometimes referred to as “ablation” by American physicians) is an outcome of the interaction between heat (or extreme cold) and tissue components; therefore, local temperature can serve as a convenient metric for monitoring the progress of thermotherapies of liver tumors. This concept has been previously implemented using thermocouple measurements and intraoperative MRI (iMRI). The translation of the local time-temperature history into the degree of local tissue thermal damage often requires the assistance of the Arrhenius thermal damage model, a model based on rate process theory. This model depends on tissue time-temperature history, a tissue-dependent frequency factor (A), and activation energy barrier (Ea). Effectiveness hinges on the accurate measurement of local time-temperature history, which is difficult to achieve with either thermocouples or iMRI. In addition, knowledge of A and Ea of tissues is largely unavailable. These limitations make temperature-based feedback control of thermotherapies of liver tumors less than optimal.

Tissue thermal damage assessments using intraoperative ultrasound (iUS) and light transmission have also been proposed previously. These approaches gauge tissue damage directly based on thermally-induced changes in tissue intrinsic sonic properties. For example, thermally-coagulated liver tissues exhibit hyper-echoic and highly scattering properties. However, the applicability of iUS is hampered by the fact that the complete thermal coagulation zone often cannot be viewed in ultrasound images as the coagulated tissue closer to the tissue surface obstructs the view of more distant tissue. In a laser-based thermotherapy, tissue thermal damage assessments using light transmission could be achieved by placing a light detector some distance away from the heating light source. The interrogated tissue volume in this setup is usually large. Because of non-uniform thermal damage within this interrogated tissue volume and the interplay of dynamic tissue optics, it is very difficult to assess tissue thermal damage (i.e., the zone of coagulation) simply based on light transmission measurements.

Optical spectroscopy, such as autofluorescence (or simply fluorescence) spectroscopy, can indicate biochemical components as well as morphological characteristics of tissue and hence theoretically may be used to directly detect tissue thermal damage. Effects of thermal damage to tissue on some optical characteristics of the tissue have been previously reported. In general, scattering coefficients have been found to increase as the degree of thermal damage of normal liver tissue increases. However, the same behavior has also been reported in metastatic liver tumors. The influences of thermal damage on other tissue optical characteristics, such as fluorescent characteristics, have yet to be reported

Moreover, none of the foregoing technologies and methods has been previously used to determine the extent (degree and volume) of thermal coagulation after the treated tissue has returned to normal body temperature.

### BRIEF SUMMARY OF THE INVENTION

Briefly stated, one aspect of the present invention is a method of identifying internal tissue of an internal organ of a patient comprising the steps of: inserting a probe into the patient and into an internal area of the organ; illuminating the internal area of the internal organ against the probe with light carried through the probe; collecting with the probe light returned from the illuminated tissue; identifying particular spectral intensity magnitude values using a light detector; and using one or more of the identified spectral values to identify the illuminated tissue as undenatured non-tumorous, undenatured tumorous or denatured tissue.

This basic method can be used to identify tumor boundaries and coagulated tissue mass boundaries within an organ such as a liver. It can be used to determine size or location or both of a tumor or a mass of thermally coagulated tissue, particularly after the coagulated tissue has returned to nominal temperature of the surrounding uncoagulated tissue of the organ. It can further be used to guide placement of a terminal coagulation instrument and to monitor the progress of coagulation around the instrument.

In another aspect, the invention is an optical apparatus for guided tumor treatment that constitutes an improvement in medical tissue ablation systems that include a tubular member configured for introduction into a patient and having one or more ablation electrodes extending therethrough and individually deployable from a distal open end of the tubular member into an ablation site within the patient, each of the ablation electrodes being coupled with an ablation energy source. The apparatus is characterized by a light detector; and at least a first optical fiber encased in a tubular needle extended through the tubular member with the one or more ablation electrodes and individually extendable from the distal open end of the tubular member into the patient at least proximal to the ablation site, the optical fiber having a first, distal end exposed to light through a distal open end of the tubular needle and a second, proximal end optically coupled with the light detector so as to deliver to the detector, light collected through the first end of the optical fiber. In a preferred embodiment the light detector is a spectrometer.

In a preferred embodiment, a second optical fiber is extended from a light source through the tubular member to a position at least proximal the ablation sight. The second optical fiber can be in the first tubular needle and in a second tubular needle. In another preferred embodiment, the light source is an ultraviolet or white light source.

The language of the claims at the end of the application is hereby incorporated by reference into the following Detailed Description.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown.

In the drawings:

FIG. 1 depicts diagrammatically the basic components of a diagnostic apparatus of the present invention for use in liver tumor treatment;

FIG. 2 depicts diagrammatically, a cross sectional arrangement of the optical fibers of the probe of FIG. 1.

FIG. 3a depicts a distribution of fluorescence (F) intensity values over time for a spectral range between about 400 nm and 750 nm;

FIG. 3b depicts a distribution of diffuse reflectance (Rd) intensity values over time for a spectral range between about 450 nm and 750 nm;

FIGS. 4a depicts the fluorescence (F) intensity values for native (untreated) and coagulated (heated treated) canine liver tissue over the range 400-750 nm;

FIGS. 4b depicts the diffuse reflectance (Rd) intensity values for native (untreated) and coagulated (heated treated) canine liver tissue over the range 500-750 nm;

FIG. 5 depicts normalized intensity values for the fluorescence ratios (F₅₁₀/F₄₈₀ and F₆₁₀/F₄₈₀) and for the diffuse reflectance (Rd₇₂₀) over time;

FIG. 6a depicts the normalized fluorescence (F) intensity for native canine liver tissue in vitro and in vivo;

FIG. 6b depicts the normalized diffuse reflectance (Rd) intensity for native canine liver tissue in vitro and in vivo;

FIG. 7a depicts the normalized fluorescence (F) intensity for coagulated (heat treated) canine liver tissue in vitro and in vivo;

FIG. 7b depicts the normalized diffuse reflectance (Rd) intensity for coagulated (heat treated) canine liver tissue in vitro and in vivo;

FIG. 8 depicts diagrammatically a second embodiment apparatus to optically locate, treat and monitor in the treatment of liver tumors, particularly tumors about two mm or more in maximum cross sectional dimension;

FIG. 9a depicts diagrammatically a typical single fiber optical probe used with the device in FIG. 8;

FIG. 9b depicts a multi-optical fiber probe also usable with the device of FIG. 8;

FIG. 10 depicts diagrammatically the remainder of the detection/monitoring diagnostic apparatus used with optical probes of FIG. 9a employing a single optical fiber;

FIG. 11 depicts diagrammatically remainder of the diagnostic apparatus used with optical probes of FIG. 9b employing multiple optical fibers;

FIG. 12 depicts diagrammatically the taking of optical measurements along five parallel axes for tumor margin detection;

FIG. 13 depicts optical diagnosis of thermal ablation using the probes of FIGS. 9a and/or 9b;

FIG. 14 depicts a conventional thermal ablation probe of the type modified for optical guidance and diagnosis;

FIG. 15 depicts one possible modification to one or more electrode “needles” of the probe of FIG. 14 to perform both optical guidance and tissue diagnosis;

FIG. 16 depicts diagrammatically a system for using the orthoscopic probe device of FIG. 14-15;

FIG. 17a depicts a method of ablation probe placement with the assistance of the optical detection system of the present invention;

FIG. 17b depicts a method of monitoring and controlling a thermal ablation procedure with the assistance of the optical diagnosis system of the present invention;

FIG. 18a depicts a distribution of fluorescence (F) intensity values over time for a spectral range between about 425 nm and about 750 nm;

FIG. 18b depicts a distribution of diffuse reflectance (Rd) intensity values over time for a spectral range between about 400 nm and about 750 nm;

FIG. 19a depicts the progress of discrete fluorescence and diffuse reflectance (Rd) spectral correlate values over time for porcine liver tissue heated to a maximum temperature of between about 60° and about 65° C.;

FIG. 19b shows the same values as FIG. 19a where the porcine liver tissue was heated to a maximum temperature of only between about 50° and about 55° C.;

FIGS. 20a and 20b are representative fluorescence (F) and diffuse reflectance (Rd) spectra from normal human liver tissue, colon metastasis tumors and cirrhotic liver tumors obtained in vitro;

FIG. 21 depicts fluorescence (F) spectra obtained from perfused and nonperfused normal liver tissue;

FIGS. 22a and 22b depict representative in vtvo fluorescence (F) and diffuse reflectance (Rd) spectra, respectively, from normal liver and primary liver tumor tissue, both perfused;

FIGS. 23a and 23b depict representative in vivo fluorescence (F) and diffuse reflectance (d) spectra, respectively, from perfused normal liver and secondary liver tumor tissue; and

FIGS. 24a and 24b depict representative in vitro fluorescence and diffuse reflectance spectra, respectively, from native and heated human colon adenocarinoma cells (SW480) in vitro.

### DETAILED DESCRIPTION OF THE INVENTION

The invention relates to a diagnostic apparatus for the identification of different tissues, particularly tumors and tumor boundaries, particularly liver tumors, and coagulated tissue masses and their boundaries, a method of using an optical diagnostic apparatus to locate tumor and coagulated tissue mass boundaries, particularly in livers, and to additionally or alternatively monitor the thermal denaturation of liver tissue as occurs during thermal ablation (coagulation), particularly of liver tumors.

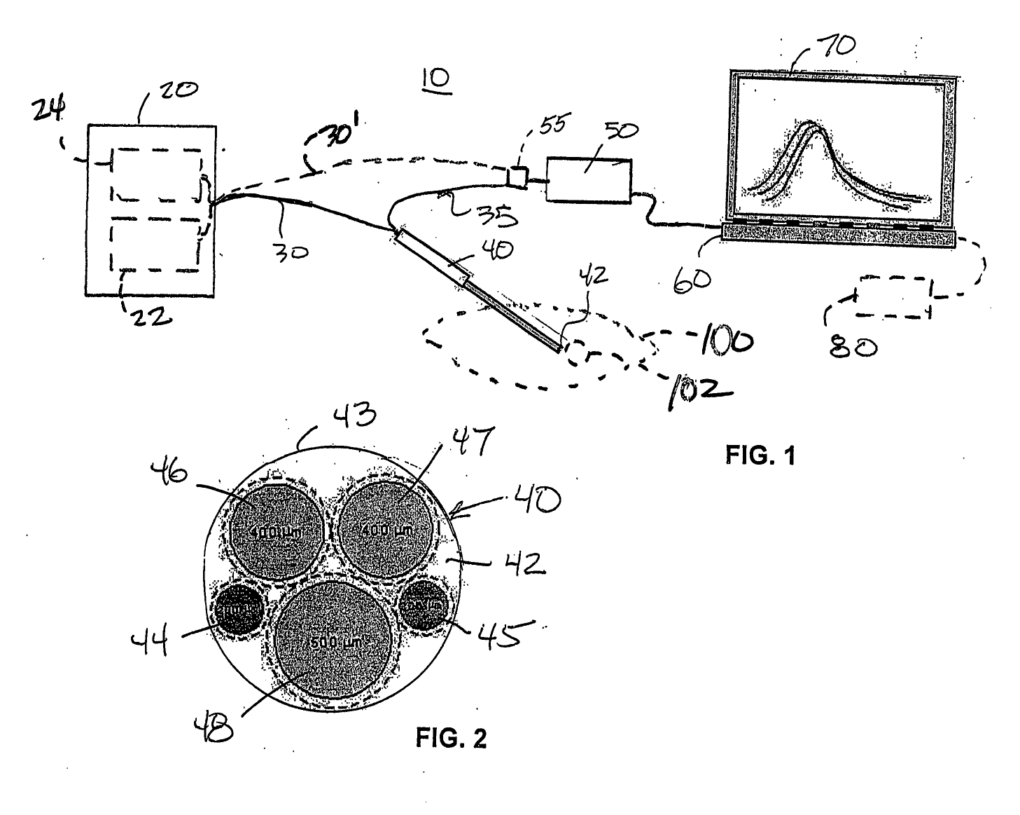

FIG. 1 depicts in diagrammatic form, the basic components of such a diagnostic apparatus indicated generally at 10. The apparatus 10 includes one or more light sources indicated generally at 20 as will be described coupled with an optical probe indicated generally at 40. Probe 40 is used to at least illuminate tissue in vivo and subsequently gather or collect light from the illuminated tissue and transfer that light to light detector 50, preferably a spectrometer 50. The spectrometer or other light detector(s) 50 provides one or more of light intensity values derived from the collected or gathered light, for one or more specific discrete wavelengths, i.e., selected individual wavelengths or discrete wavelength ranges (e.g. 1 nm or 10 nm, respectively). A computer 60 uses one or more algorithms to analyze this spectral data to determine whether the tissue being illuminated is tumorous or non-tumorous, coagulated or uncoagulated, in particular, coagulated by thermal exposure. The tissue is monitored to a sufficient degree of denaturation to cause necrosis of the tumor cells.

The light source(s) 20 includes at least a source of light at a frequency or in a frequency range selected to induce autofluorescence in the tissue illuminated by the light and further preferably includes a source of light suitable to generate diffuse reflectance in tissue illuminated by that light. More particularly, the suggested autofluorescence (hereinafter also referred to simply as “fluorescence”) inducing light is generally in the ultraviolet (“UV”) spectrum, more particularly between about 320 nm and about 360 nm. In one preferred embodiment, the fluorescence inducing light source 22 is a 337 nm nitrogen dye laser operated, for example, at a repetition rate of about 20 Hz with an average pulse energy of about 50 μJ at the illuminated tissue surface. Other fluorescence inducing light sources such as UV lights or mercury vapor lamps or broadband lasers may be suitable. The diffuse reflectance light source 24 is sufficiently bright to generate enough diffuse reflectance in the illuminated liver tissue to identify changes in the diffuse reflectance over time, at least in the spectrum region(s) of interest. For example, a one hundred fifty (150) watt halogen lamp may be used as source 24 to provide light over the entire visible and deep red/near infrared region of about 400 nm to at least about 750 nm and up to about 850 nm, if desired. All diffuse reflectance data departed herein has been generated with a 150 watt halogen light source (Fiber Light, Model 180, Edmund Industrial Optics, Barrington, N.J.), which has been found to provide a very stable output (about one percent or less variation). Alternatively, a single broadband light source might be provided, for example, with a narrowband pass filter to generate both white or other diffuse reflectance light and ultraviolet light for the reasons stated above. Fluorescence and diffuse reflectance reference measurements can be taken using, for example, a fluorescence standard (Rhodamine 6G in ethylene glycol, 2 mg/L) and a diffuse reflectance standard (20% reflectance plate, Labsphere, North Shutton, N.H.) to evaluate instrument performance between uses.

The probe 40 is coupled with the light source(s) 20 through an optic cable 30 including at least one optical fiber carrying light from the source 20 to a working tip 42 at the distal end of the probe 40. The probe 40 is sufficiently long yet sufficiently thin to be inserted directly into the liver without permanent damage to the liver. Light is gathered or collected from the internally illuminated liver tissue and is carried to a spectrometer 50. The fiber optic cable 30 from the source(s) 20 could be branched as indicated in phantom at 30′ from its coupling with the light source(s) 20, to carry gathered light to the spectrometer 50 along the same optical fibers in the probe 40 used to transmit the illuminating light More preferably, a separate fiber optic cable 35 is provided from the probe 40 so that tissue can be illuminated and diffuse reflectance light collected or gathered simultaneously from the illuminated liver tissue and directed to the spectrometer 50. In this embodiment, each cable 30 and 35 contains at least one and preferably more than one optical fiber dedicated, to carrying illuminating light from the light source(s) 20 (in cable 30) to the working tip 42 or collecting light from the illuminated tissue at one or more locations directly opposite or about the working tip 42 of the probe 40 (via cable 35). Thus, the working tip 42 is both light emitting and optical sensing.

FIG. 2 depicts one possible arrangement where the probe 40 comprises a pointed, seventeen gauge stainless steel hollow tube (“cannula”) 43 encasing five separate shielded optical fibers 44-48. The smallest diameter 200 μm fibers 44, 45 are dedicated to fluorescence inducing UV and diffuse reflectance (e.g., white) light, respectively, while the remaining three fibers 46-48 (two 400 μm and one 500 μm) are dedicated to collecting light from the illuminated tissue at and around the working (distal) end 42 of probe 40.

Optionally, a band pass filter, for example, a long pass filter with a 380 nm cutoff indicated in phantom at 55, can be provided between the probe 40 and spectrometer 50 to eliminate at least one end or, if desired, both ends of the light spectrum range that are not of interest as well as any stray light from the UV and white light source. The spectral range of interest for autofluorescence (F) and diffuse reflectance (Rd) has been found to be between about 380 nm and about 850 nm. Furthermore, the spectrometer 50 may be provided with an entrance port slit to yield a desired spectral resolution. For example, a two hundred μm slot yielded a spectral segment resolution of about 10 nm with an S2000-FL spectrometer from Ocean Optics, Dunedin, Fla. Alternatively, the light detector 50 might be provided with one or more photocells, charge coupled arrays or other light detecting devices and one or more narrow band light filters used with the individual detector(s) to directly measure intensity magnitude at a particular wavelength or narrow range of wavelengths and thereafter digitize those values so that they can be used directly by the computer 60.

The computer 60 is coupled to the light detector 50 by suitable means such as an A/D converter and an appropriate data channel and is used to both control the operation of the light detector 50 and to analyze the spectral data outputted by the light detector 50. The computer 60 can be any type of ordinary laptop or personal computer. Software for controlling the operation of commercial spectrometers is typically provided with such devices. The computer 60 is programmed with algorithms using spectral data from the light gathered by the probe 40 to distinguish between tumorous and non-tumorous tissues and between native and denatured tissues, particularly liver tissues. Output from the computer 60 can be displayed to the user by suitable means such as a monitor 70 and/or routed to one or more other devices, indicated diagrammatically in phantom at 80, for example, a computer peripheral such as a printer or a controller of a thermal ablation probe (not depicted in FIG. 1), to automatically terminate the ablation treatment.

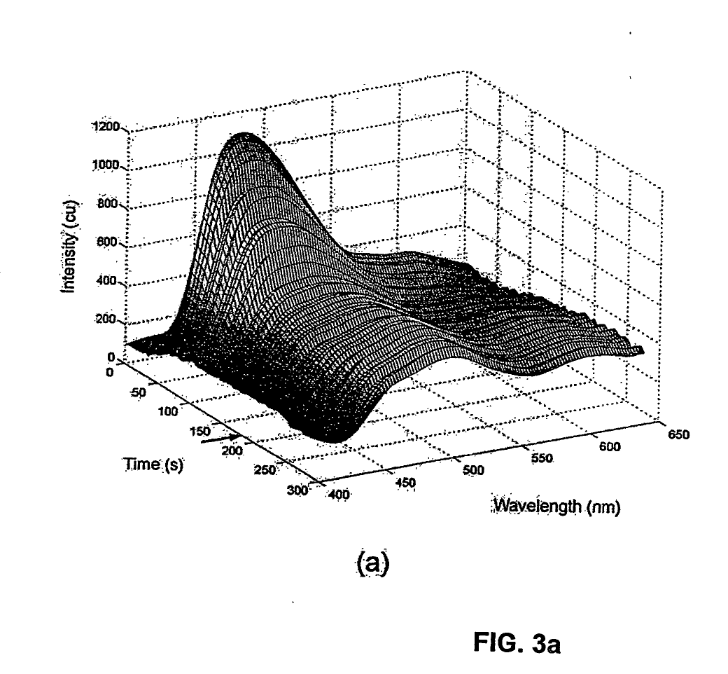

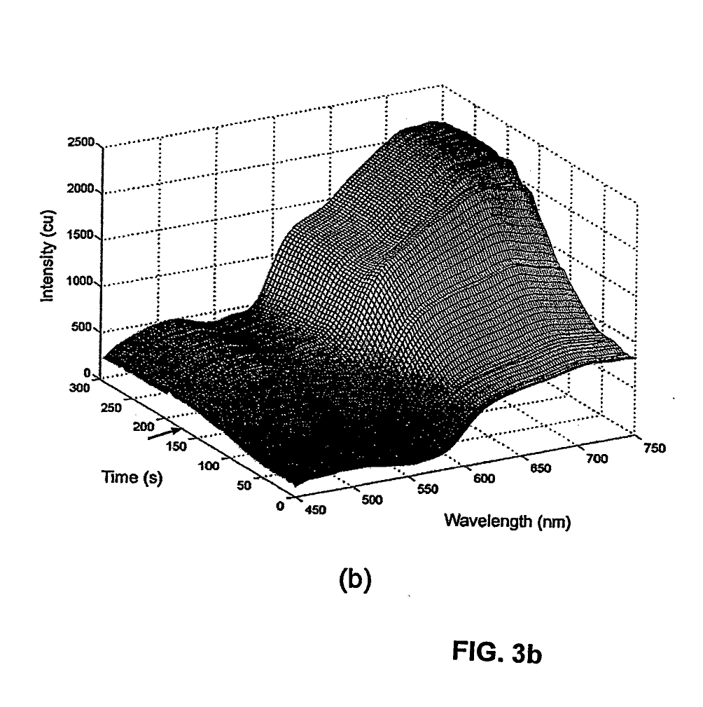

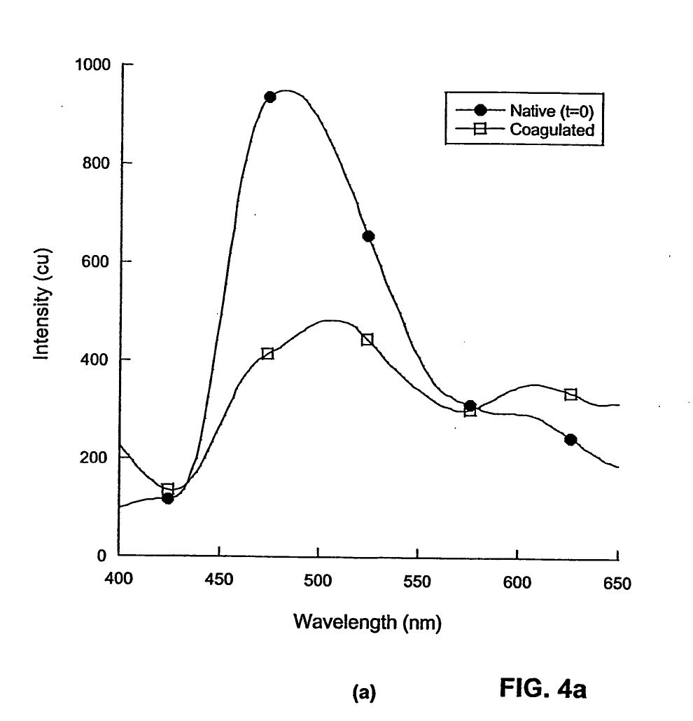

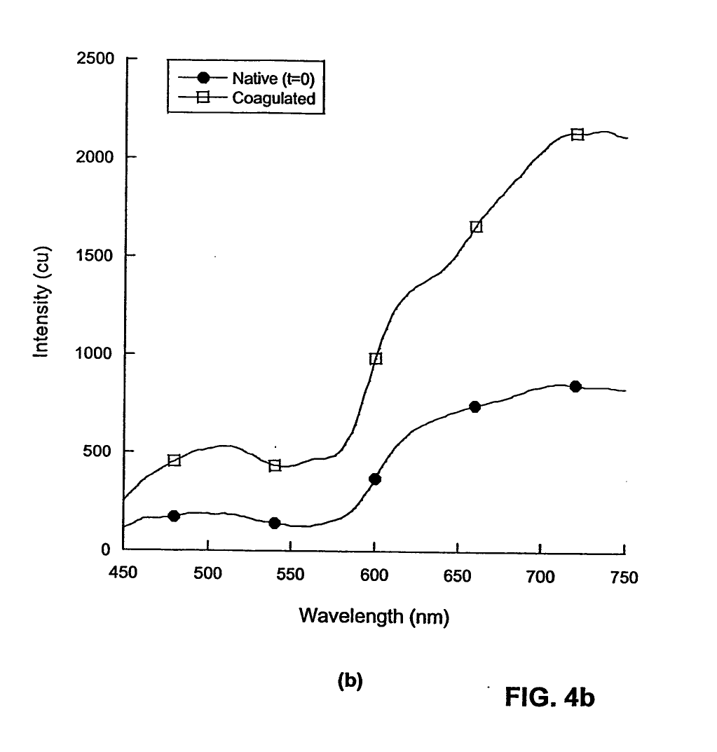

FIGS. 3-5 depict various spectral characteristics of canine liver tissue. In particular, FIGS. 3a and 3b depict time course plots of autofluorescence (F) and diffuse reflectance (d) spectra from continued heating of the observed tissue. The corresponding initial (i.e., native) and final (i.e., heat coagulated) intensity values for the fluorescence spectra are plotted in FIG. 4a while those of the diffuse reflectance spectra (Rd) are plotted in FIG. 4b. The advance of thermal damage gradually induces several alterations in both fluorescence and diffuse reflectance spectra from liver tissue. It was found that peak fluorescent intensity initially occurred at about 480 nm and decreased by 45% in magnitude over the indicated heating period. During that time, the peak intensity location also shifted to about 510 nm. In FIG. 3b, diffuse reflectance was at a maximum between about 700 nm and about 750 nm, initially peaking between about 710 nm and about 720 nm, and substantially maintaining that peak throughout the time period (five minutes). Over that time, diffuse reflectance intensity between 600 nm and 750 nm increased anywhere from 110% to 165%. Several other line shaped changes were noted as well. A depression appeared in the diffuse reflectance signal between about 600 nm and about 700 nm at around the same time that peak intensity was achieved. Furthermore, a shoulder appearing at about 610 nm became more pronounced as heating continued. However, the intensity around the shoulder remains relatively stable (standard deviation equaled 12% of mean intensity).

FIG. 4a depicts autofluorescence spectra of native (unheated) and denatured heat coagulated) canine liver tissues. FIG. 4b depicts diffuse reflectance spectra for such tissues before and after the indicated heating cycles. Two representative spectral intensity valves or “spectral correlates” for fluorescence indications of thermal damages are defined. There is a significant reduction of peak intensity value as heating progressed. Therefore one spectral correlate from the fluorescence spectrum over time is the change in the intensity value of what is the peak spectral value in the initial (native) tissue, here about 480 nm. The normalized inverse of those intensities over time would increase significantly. There is also a relative increase in spectral intensity at about 600 nm, more particularly, about 610 nm in the indicated data. Since this change is in an opposite direction to that of the peak intensity at about 480 nm, a ratio of the two, i.e. F₆₁₀/F₄₈₀ would provide an intensity value with even greater increase over time with the denaturation of the tissue. A ratio correlate tends to eliminate systemic bias in the equipment and irregularities in the light outputs as well as variations from subject to subject. Separately, the peak intensity frequency shift can also be represented by a ratio of the fluorescence intensity of the spectral component expected to have the peak intensity value at the end of the denaturation (heating) process to the inverse of the initial peak intensity value, i.e. F₅₁₀/F₄₈₀.

The most prominent spectral change in diffuse reflectance (Rd) is the increase in intensity, particularly at wavelengths of about 700 nm and above. There was a relative increase as well at about 500 nm and a general increase of all intensities above about 625 nm. Some of this difference above and below about 600 nm may be due to the optical absorption of blood. Optical absorption between about 600 nm and about 750 nm is one to two orders of magnitude less than the degree of absorption between about 450 nm and about 600 nm. Therefore, the selection of a wavelength in a region of relatively weaker blood absorption allows for the detection of changes presumably related to the damage of hepatic tissue rather than damaged blood or changes in profusion or oxygenation. FIG. 4b indicates that the greatest magnitude in diffuse reflectance intensity occurs between about 700 nm and about 750 nm as does the greatest change in intensity. Accordingly, any spectra intensity segment above about 600 nm and suggestedly in the range of about 700 nm to about 750 nm can be used to monitor this type of thermal denaturation. For the canine tissues examined, the statistical maximum was found to be at about 720 nm.

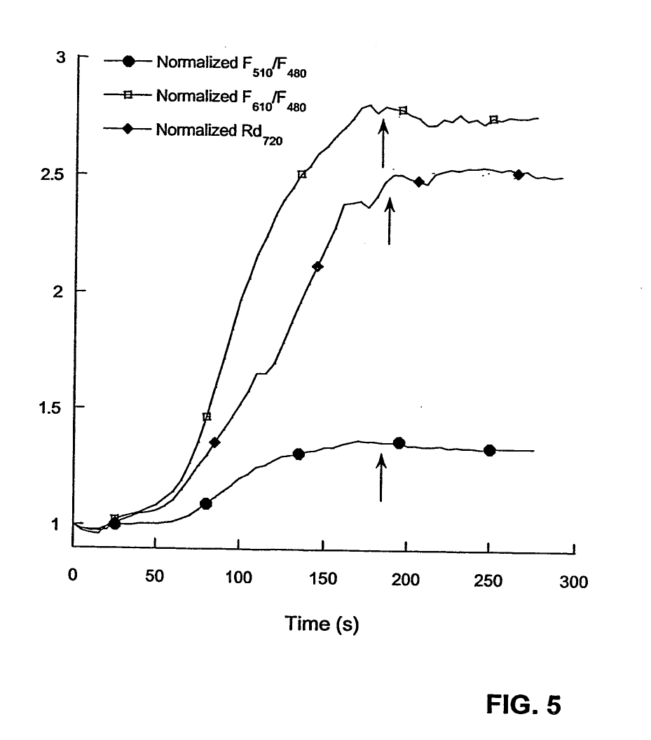

FIG. 5 depicts the values of the aforesaid three spectral correlates: the autofluorescence ratios F₅₁₀/F₄₈₀, F₆₁₀/F₄₈₀ as well as the intensity maximum of Rd₇₂₀. All are normalized with respect to their initial (native tissue) values. It can be seen from the figure that the histories of all three spectra correlates relate to thermal damage in at least two distinct phases: an initial ramp-up phase followed by a rapid increase phase. Each of the three correlates exhibits a third, plateau phase, where the tissue was heated to a maximum temperature between about 60° C. and about 70° C. The spectral correlates plateaued prior to the termination of heating where the tissue was fully denatured. This plateau phenomena, however, was not observed in cases of “partial denaturation”, i.e. where the tissue was heated to only about 50° C. The dynamic range of the three identified normalized spectral correlates (maximum value−minimum value) varied from one another. As indicated in FIG. 5, normalized F₆₁₀/F₄₈₀ has a dynamic range in this instance that was about five times that of normalized F₅₁₀/F₄₈₀, even though both reached a stable plateau at about the same time.

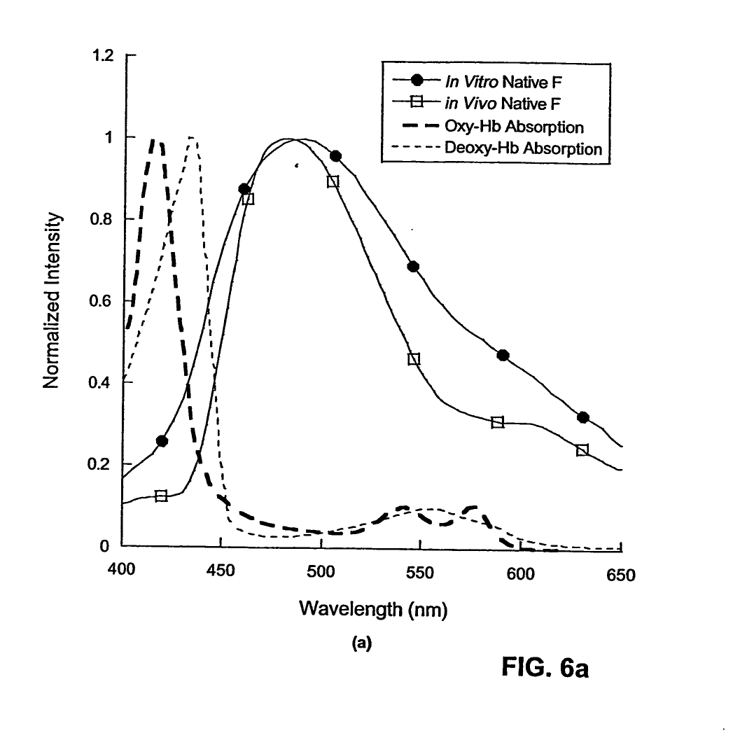

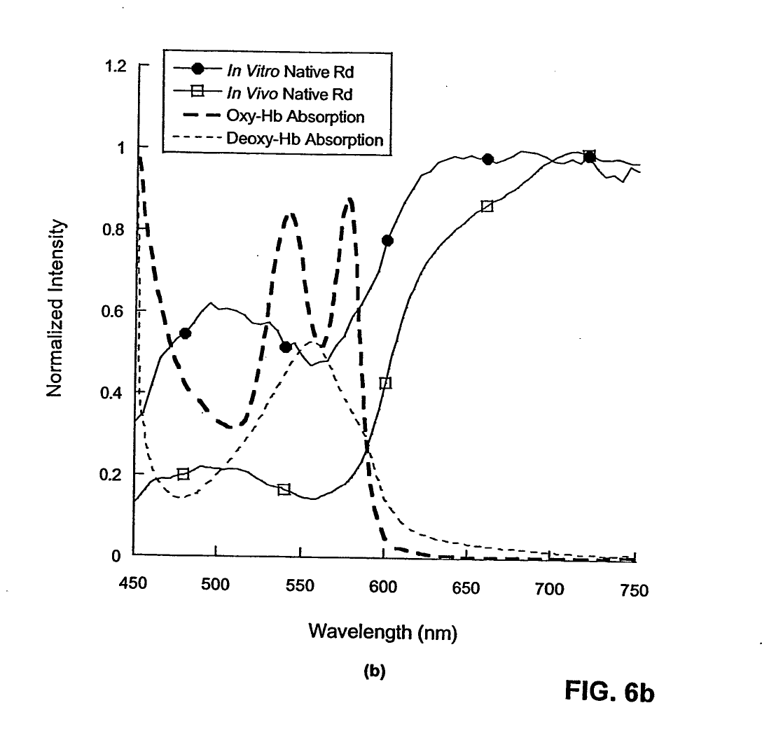

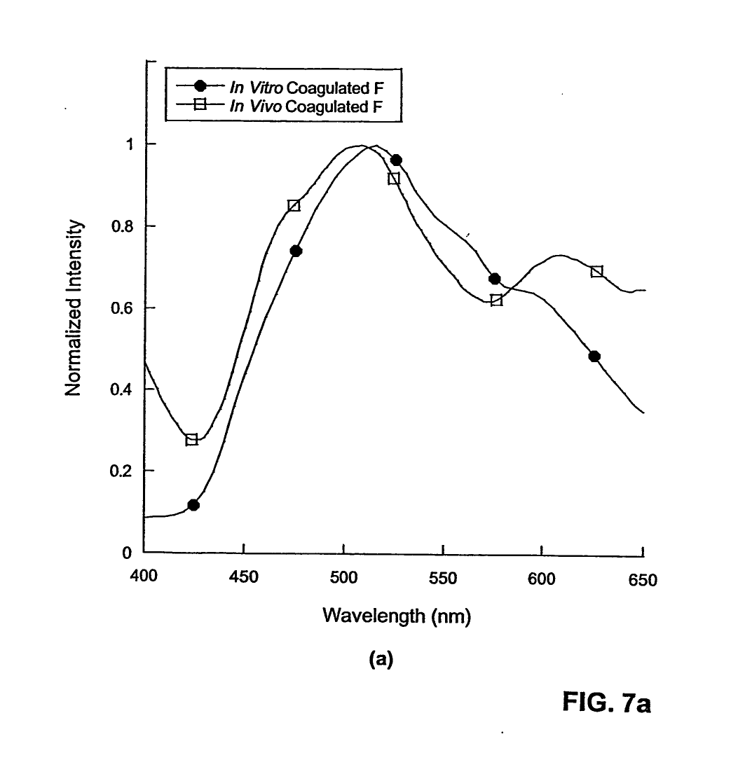

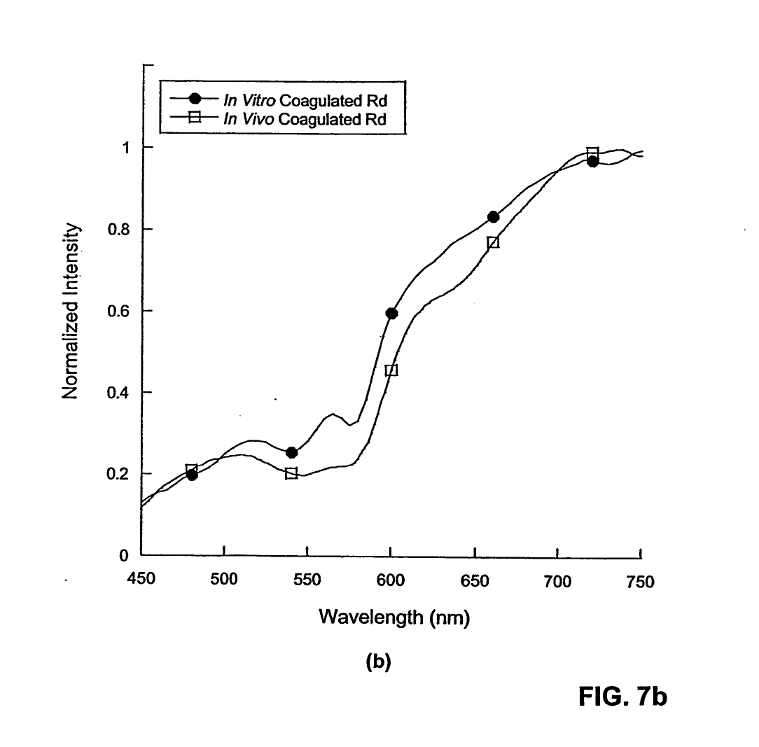

FIGS. 6a and 6b show comparative line-shape intensity values for fluorescence (F) and diffuse reflectance (Rd), respectively, of native (undamaged) in vitro and in vivo canine liver tissue normalized to the maximum measured value for all spectra. Similar fluorescence and diffuse reflectance spectra profiles are provided in FIGS. 7a and 7b, respectively, for fully denatured (coagulated) canine liver tissue, in vitro and in vivo. These show a relative correlation in both peak locations at both ends of the denaturation process. To help understand the differences between the native in vitro and in vivo spectra, absorption spectra of oxy-deoxy hemoglobin were plotted along with native liver spectra values. While the native fluorescence spectra in FIG. 6a were found to have similar peak locations (about 490 nm in vitro, about 480 nm in vivo), the full width half maximum (“FWHM”) intensity value of the in vivo fluorescence peak was about 90 nm as compared to a FWHM of 145 nm for the in vitro peak. The major difference between native diffuse reflectance spectra in FIG. 6b was a more pronounced valley between about 500 and 600 nm observed in vivo. A noticeable difference in line-shape was found between the fully denatured fluorescence spectra in FIG. 7a: the in vivo curve shows a secondary peak at about 600 nm. As for the fully denatured diffuse reflectance spectra in FIG. 7b, the in vivo curve exhibits a valley at about 650 nm not seen in the in vitro curve.

Reasonably strong correlation was found between each of the F₅₁₀/F₄₈₀, F₆₁₀/F₄₈₀, and Rd₇₂₀ correlates and the actual thermal damage observed in the tissue and represented well-understood histological markers of tissue thermal damage such as hemorrhage and blood coagulation. Additionally, analysis indicates that these three spectral correlates of thermal damage are able to differentiate between various histological grades of thermal damage with a reasonable level of confidence. Each of the fluorescence correlate ratios appear to be able to differentiate between intermediate levels of tissue damage and undamaged or low levels of damage better than the diffuse reflectance correlate Rd₇₂₀, at least in the samples studied to date. Post ablation examination of tissues indicates that in complete denaturation, the spectral correlates do reach their plateau values. Therefore, in addition to actually monitoring their values, their time varying (time derivative) values can also be monitored as a correlate. In each case, the intensity correlate and its upward ramping, time derivative will peak. The time derivative then drops to zero, nearly zero, or even below zero at the plateau. Heating tissue to the plateau level or into the plateau level assures complete denaturation of the tissue.

System performance considerations are also a factor in selecting appropriate correlates to monitor tissue to thermal denaturation. The pulse to pulse energy variation in laser pulses (about fifteen percent of the mean energy value) encouraged the use of ratio correlates. However, between about 500 nm and about 750 nm, the standard deviation of intensity of the halogen lamp white light source was found to be less than one percent This permitted the use of diffuse reflectance intensity at a single wavelength in the 700-750 nm range to monitor changes. Furthermore, combination of poor spectrometer sensitivity and weak halogen lamp emissivity in the near-infrared region led to poor signal to noise ratios beyond about 750 nm. Accordingly, the about 720 nm initial peak was more stable and usable.

The observed increase in diffuse reflectance intensity can be explained by thermally-induced changes in tissue optical properties. The dominant change in liver tissue optical properties upon thermal coagulation is an increase in the reduced scattering coefficient (μₛ′). This increase reflects changes at cellular and intracellular levels such as protein denaturation, hyalinization of collagen, cytoskeleton collapse and cell membrane rupture, at least some of which are known to occur at onset temperatures of between about 45° and about 90° C. These all affect the size and distribution of scattering particles in the tissue and, consequently, light distribution.

The decrease in peak fluorescence intensity can be explained by several factors. First, there is a decrease in penetration depth and local fluence rate at the excitation wavelength (about 330 nm). Penetration depths for native and thermally coagulated liver tissue are estimated to be about 0.17 nm and about 0.12 nm, respectively. This leads to a decrease in the volume of tissue being illuminated. With a uniform distribution of fluorophores, the reduction of illuminated volume translates into a decrease in the total fluorescence emission. Second, thermal damage leads to a decrease in the fluence rate of excitation light under the collection fibers and a consequent decrease in fluorescence intensity. Third, there is a degradation and quantity reduction of the fluorophores. It is believed that autofluorescence at 337 nm excitation is provided primarily by collagen, nicotinamade adenine dinucleotide (NADH), and flaven adenine dinucleotide (AD). Fluorescence of protein is due to interaction of photons of specific energy with specific chemical bond (e.g., UV photons with collagen crosslinks). Thermal denaturation of proteins breaks the bonds responsible for their autofluorescence properties. The interstitial extracellular matrix and liver tissue is known to contain ten different types of collagen including fibrillar collagens, such as collagen I. The fluorescence emission peak of collagen I of about 410 nm (337 nm excitation) decreases dramatically as a function of thermal damage. Furthermore, thermal injury alters the bio-physiological function of tissue and destroys micro-organelle (e.g., mitochondria) at a microscopic level, which would lead to reduction of NADH and FAD quantity in cells and hence fluorescent intensity between about 400 nm and about 550 nm emissions. Therefore, the observed decrease in overall fluorescence intensity, as well as the shift in peak to longer wavelengths, appears to be a combined effect of thermally-induced changes in tissue optics and degradation/quantity reduction of fluorophores.

It is further believed that the observed differences in line-shape between in vitro and in vivo native and denatured liver tissue spectra can be primarily attributed to increased blood absorption in vivo, particularly by hemoglobin. The compression along the wavelength axis of the native in vivo fluorescence spectrum relative to the corresponding in vitro spectrum can be explained by the strong absorption peaks of oxy-hemoglobin and deoxy-hemoglobin at 413 and 432 nm, respectively, and the relatively weaker absorption peaks between about 540 nm and about 580 nm (FIG. 6a). Increased blood absorption between about 540 nm and about 580 nm also explains the valley in this region in the native in vivo diffuse reflectance spectrum (FIG. 6b).

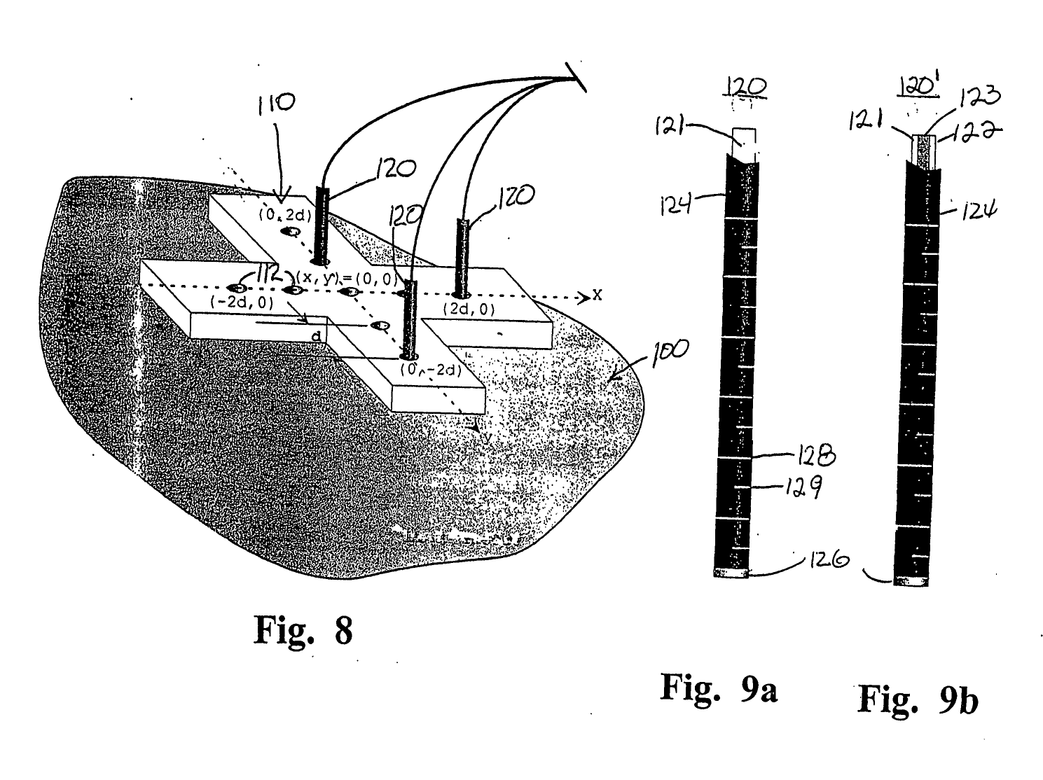

FIG. 8 depicts the tissue contacting portions of a more specific diagnostic system used to identify the location of a liver tumor before treatment or a mass of coagulated tissue after treatment. It can further be used for the positioning of a thermal coagulation device to about the geometric center of the tumor to destroy the tumor. The system can be further used to monitor thermal coagulation of the liver tissue around the tumor during treatment to determine when the coagulated treatment is completed or should be concluded. In FIG. 8, the portion of the system applied to the liver 100 may include a probe anchor 110 and a plurality of individual optical probes 120. The depicted anchor 110 is in the form of a cross but it might have other forms, e.g., a circle or rectangle, and has a two-dimensional grid of openings sized to receive the probes being used. If used, the anchor 110 is physically secured to the surface of the liver 100 by suitable means such as pins (not depicted) on its lower side. The anchor 110 is used to register the relative radial position and depth of optical probes 120 and/or 120′, which are inserted into the liver 100. The anchor 110 is provided with a plurality of preferably regularly spaced openings 112 through which probes 120, 120′ can be slid. The height of the anchor 110 and relative dimensions of the openings 112 to the outside of the probes 120, 120′ are such that each probe 120, 120′ maintains an orientation and position coaxial to the center line of the opening 112 through which it passes. Referring to FIGS. 9a and 9b, each optical probe 120, 120′ can contain one optical fiber (121 in 120) or more optical fibers (121, 122, 123 in 120′). The optical fibers are passed through a tubular member 124 made of any suitable, sterilizable material such as stainless steel, to a window 126, made of suitable transparent, sterilizable material such as quartz or an appropriate, clear synthetic polymeric plastic, for example, at the distal end of the shell 124. Preferably, regularly spaced scale marks 128, 129 can be provided along the length of the shell 124 so that the relative depth of the probe 120 in the anchor 110 can be visually determined by the operator.

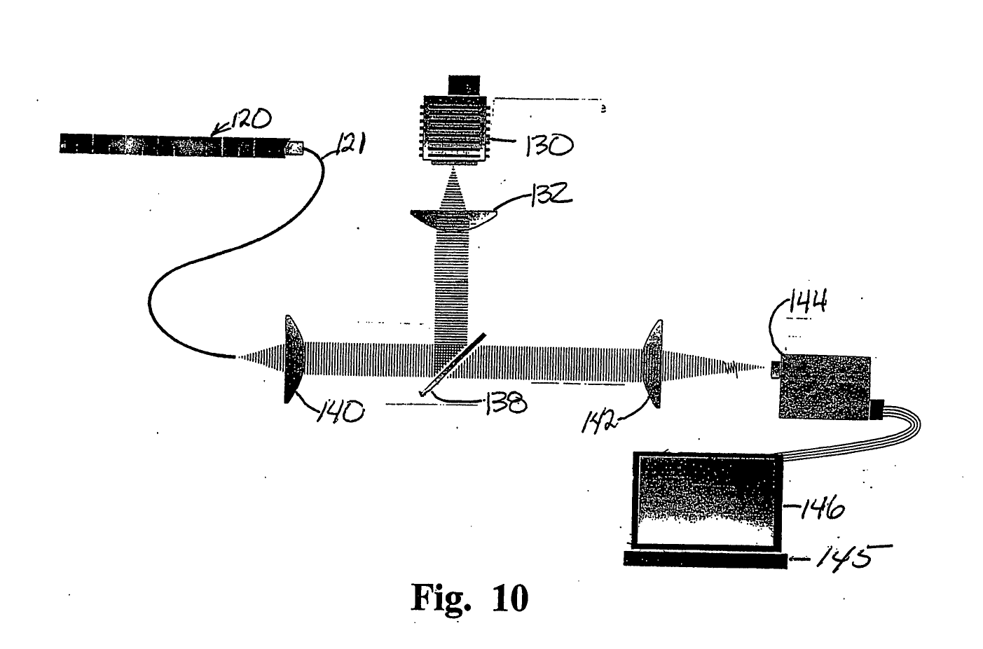

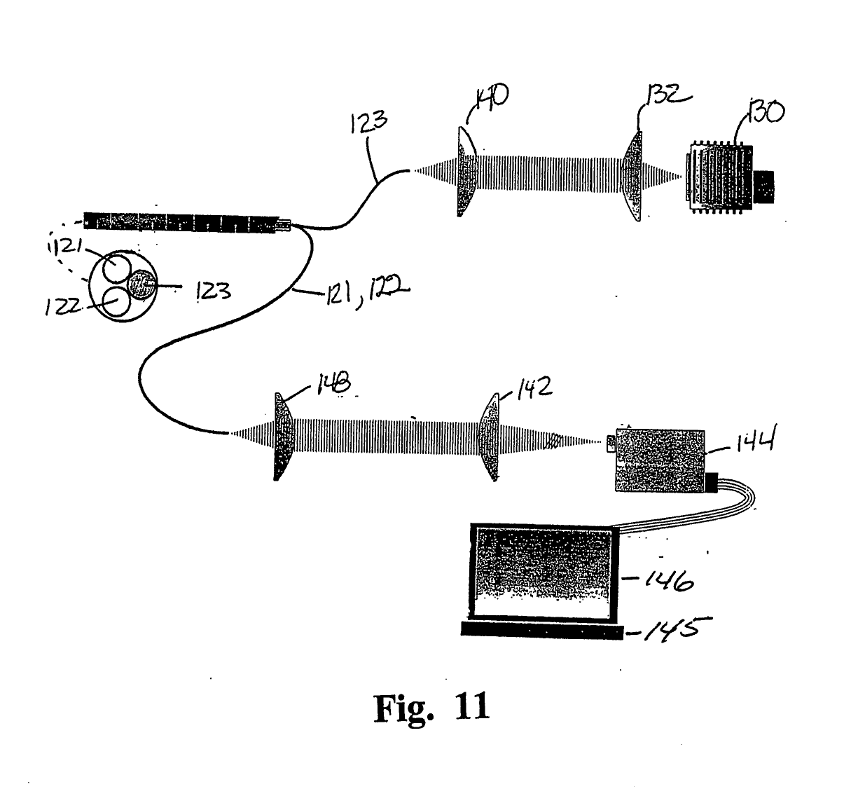

The optical probes 120 and/or 120′ are coupled with remaining components of a system indicated in FIG. 10 or 11, respectively. Where optical probes 120 having a single optical fiber 121 are provided as shown in FIG. 10, the remainder of the system includes the one or more light sources indicated collectively at 130, each outputting a beam of light along a light path such as through a collimating lens 132, a dichroic element [e.g. mirror] 138 and focusing lens 140 where the light is fed into the proximal end of the optical fiber 121. Light gathered from the illuminated or excited subject tissue is passed from the tip of the optical fiber 121 back along another light path including the lens 140, dichroic element [e.g. mirror] 138, through element 138 and through focusing lens 142, where it is fed into a light detector, preferably a spectrometer 144. The output of the spectrometer 144 is the collection of spectral intensities of the autofluorescent light (i.e. F(λ)'s) and/or the diffuse reflectance light (i.e. Rd (λ)'s) collected by the probe 120 or 120′, which is fed to a computer 145 for processing, analysis and display on device 146. FIG. 11 depicts one suitable system that is shown, for example, as being used with one or more probes 120′ containing multiple optical fibers. One or more fibers can be dedicated to providing light from the source(s) 130 and the remainder can be dedicated to gathering light from the illuminated tissue. Probes 120′ are more desirable at least for diffuse reflectance measurements where it is best to illuminate the tissue while simultaneously gathering or collecting diffuse reflectance light from the illuminated tissue. Such a configuration also allows for the elimination and consequent transmission loss of the dichroic element [e.g. mirror] 138 but requires an additional lens 148. A single optical fiber probe 120 is satisfactory for autofluorescent measurements where the tissue must first be illuminated with the excitation light and fluorescence occurs after the excitation light is removed.

The system depicted in FIG. 10 operates by illuminating tissue opposite the window 126 with light source(s) 130 through each optical fiber 121 and collecting or gathering light returned from the illuminated liver tissue proximal the probe window 126 (e.g. autofluorescence or diffuse reflectance) and delivering that collected light to the spectrometer 144. In the system of FIG. 11, light can be continuously supplied from source 130 along one of the optical fibers 123 and collected or gathered by the remaining fibers 121, 122 from the immediately surrounding tissue to be carried to the spectrometer 144. Where multiple light sources are needed or desired, it may be necessary to physically cycle the light sources before the collimating light lens 132 or to introduce a suitable light path such as by means of another dichroic element [e.g. mirror], to direct light from multiple sources to the collimating light element 132 or focusing lens 140. Alternatively a broad spectrum light source (e.g. white light) could be provided and appropriate filters interposed to pass the desired band(s) of light frequencies.

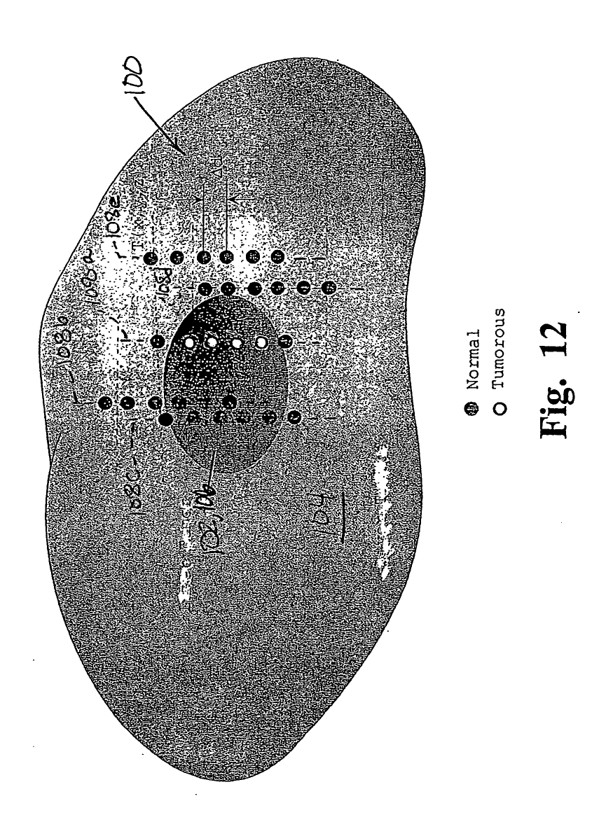

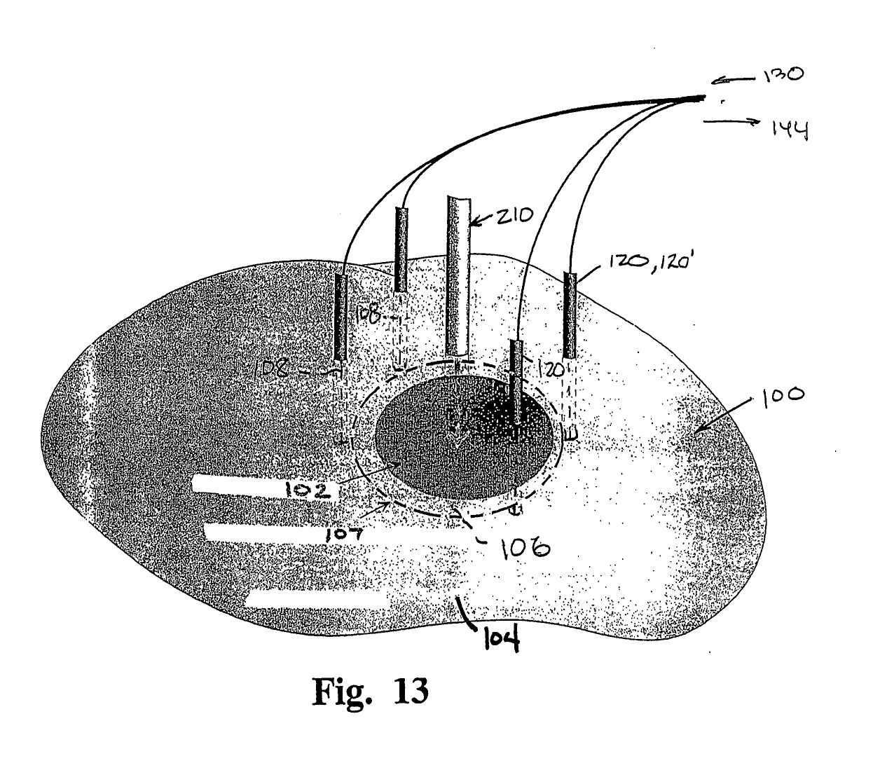

Use of either system of FIGS. 10 and 11 will now be described with respect to FIGS. 8, 12 and 13. Initially, a non-invasive detector (e.g. ultrasound, iNMR) is used to identify the approximate location of the liver tumor and to position the probe anchor 110 centered as directly over the tumor as such observation of the tumor permits. After the anchor 110 is secured, one or more optical probes 120, 120′ are inserted into and pushed through one or more holes 112 of the probe anchor and into the liver tissue. The probes are advanced along individual paths 108a-108e into the tissue. At spaced locations along each path, optical readings of the liver tissue at the window end 126 of the probe(s) are taken, preferably at regular intervals. In particular, after the anchor 110 is secured, each optical probe 120, 120′ will be inserted into and pushed through one of the anchor holes 112 until its tip 126 reaches the surface of the liver 100. At this point, the position of the probe tip, P(x, y, z), which is determined by the hole location (x, y) and the scale markings 128, 129 on the side of the probe (z), will be recorded and an initial set of optical spectra [OP(λ's)] will be acquired from the contacted liver tissue. Once the initial position registration of the probe and spectral recordings of the liver surface at the (x, y) position are completed, the optical probes 120, 120′ will be pushed into the liver tissue by a fixed distance Δd, which suggestedly is no more than about one-fifth of the estimated diameter of the tumor. At this point, a new set of optical spectra will be acquired by repeating the illuminating and collecting steps and the tip location of the optical probe registered. This positioning and interrogating procedure can be continued until the probe tip moves along its path into contact with the apparent edge of the tumor and/or through the tumor and beyond its far edge, or past the tumor located at other locations by the probe or other diagnostic means. The same recording procedure will be performed at each other desired hole 112 location. The acquired optical spectra [OP (x, y, z, λ)] is analyzed by the computer 145 to determine tissue characteristics T (x, y, z) at corresponding locations P (x, y, z). The tissue characteristics, tumorous and normal or “native” (i.e., non-tumorous), can be expressed visually in three dimensions in the form of a series of multicolored or variably shaded points on a visual display 146 as shown to illustrate and distinguish the tumor data points and non-tumor data points. Based upon the collection of data points, the volumetric center of the tumor will be determined. Guided by this information, a thermal probe 210 will be deployed into the center of the tumor and denaturization of the tumor by thermal coagulation will be carried out. This is illustrated diagrammatically in FIG. 13 where, for instance, a radio frequency ablation (“RFA”) or other thermal ablation probe 210 is passed through one of the openings 112 of the anchor 110 in FIG. 8 to a suitable depth to position a working end of the probe in the liver tumor at or proximal to its center. Optical probes 120, 120′ can be positioned through the anchor 110 around the measured and interpolated margins of the tumor so as to define a boundary to which the liver tumor and surrounding tissue are coagulated. It should be noted that multiple optical probes also may be used simultaneously in the beginning of the process (i.e. FIG. 12) when the investigated tissue volume is large in order to reduce the time for locating the volumetric center of the tumor. It should further be appreciated that the anchor 110 is a convenience, not a necessity. Individual optical and RF probes can be generally positioned by ultrasound, iNMR or a frameless surgical navigational system like that disclosed in U.S. Pat. No. 6,584,339, incorporated by reference herein, and the various probes 120, 120′, 210 positioned manually, without an anchor, guided only by such instruments and optical readings from the probes themselves. It should be appreciated that the same equipment can be used and the same steps followed to identify the location of a mass of coagulated tissue in a surrounding body of uncoagulated tissue of the organ.

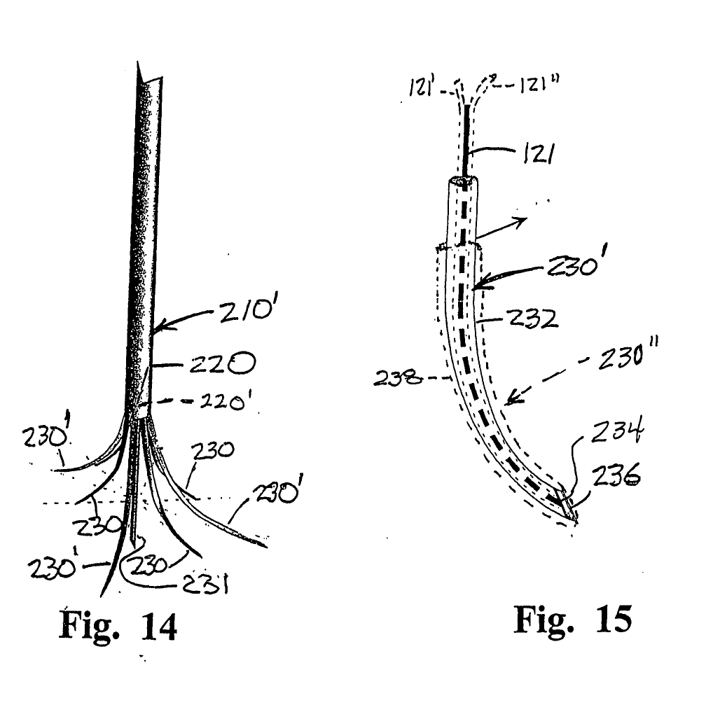

FIG. 14 depicts diagrammatic one type of thermal coagulation (ablation) device, an orthoscopic, radio frequency (“RF”) probe 210, which includes a hollow outer tube or cannula 220 through which are inserted a plurality of individual conductive electrode elements or “needles” 230. The tip of the cannula 220 can be blunt as indicated in solid lines or have a tapered tip as indicated in a phantom line at 220′ for easier penetration. The needles 230 are preferably metallic to carry radio frequency energy applied to them. The probe 230 may also be provided with an extendable, active trocar tip 231. Probes of this type are currently supplied by manufacturers such as RITA Medical Systems, Inc. of Mountainview, Calif.

The RF probe 210 is modified to function as a tissue diagnostic device as follows. One or more of the conventional needles 230 is replaced with a modified needle 230′ or 230″ as shown in FIG. 15. Each of the modified needles 230′, 230″ includes a tubular body 232 sufficiently rigid to penetrate the organ tissue without significant deflection from a predetermined shape, preferably curved. At least a first optical fiber 121 is passed through the hollow interior of the tubular body 232 to an open, distal end 234 of the tube 232 where a window 236 of quartz, glass or a suitably heat resistant and transparent synthetic polymeric plastic is provided. Alternatively, in a needle 230″, a clear transparent synthetic polymeric plastic coating or layer indicated in phantom at 238 is provided over the distal end of the tube 232 to replace the window 236 and extended up a length of the tube 232. Coating or layer 238 thereby closes the open end 234 and protects the optical fiber 121 and further insulates the distal end of the tube 232 from the surrounding tissue to prevent coagulation from that end of the tube. The coating 238 could extend the entire exposed length of the tube 232 or along just so much of the distal end of the tube 232 as needed to provide a desired distance of the open end 234 from the active coagulation volume. Hollow needles 232′ are already known and used in such devices to deliver fluids. At least a first fiber 121 and possibly a second 121′ or even a third 121″ (both in phantom) might be passed through the bore of tube 232 to the distal open end 234.

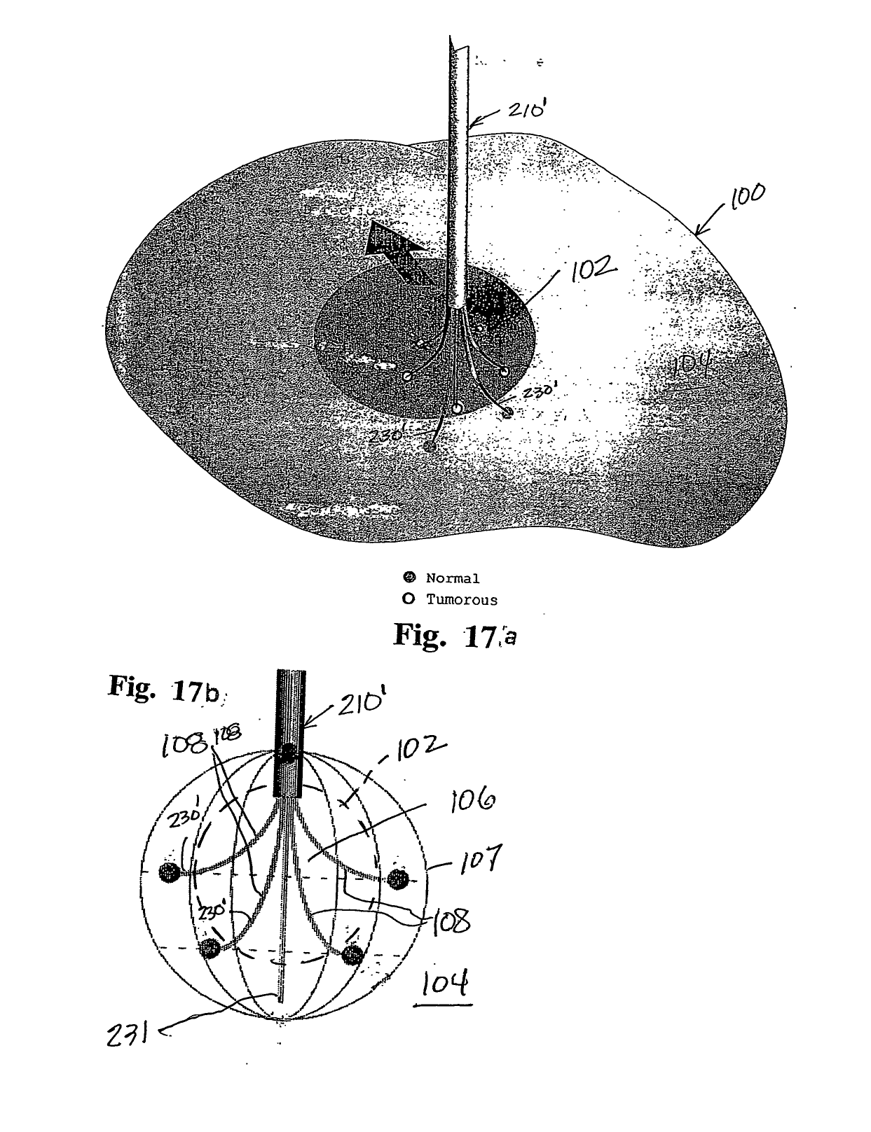

Each needle 230′, 230″ can be individually positioned in the same way as each other electrode 230 so as to take a series of measurements as described above with respect to the first embodiment device described in FIGS. 8-12 to optically locate a tumor and position the distal end of the tube 220 and the array of electrodes 230. The relative radial position (x, y) of each optical fiber tip (and each electrode) from the end of the cannula 220 is usually determined by the needle structure of the probe 210′ whereas the axial position (z) is usually determined by the insertion depth of the needle 230′ through the cannula 220. The electrodes and needles 230, 230′, 230″ can be arrayed in a common plane as indicated in FIG. 17a, or in a series of planes or other locations spaced generally about a sphere centered around the heating center of the probe 210′. The probe 210′ is generally supplied in different sizes which can be used on tumors up to about 7 cm in diameter, which is approximately the maximum diameter of the sphere which represents the coagulation boundary 107 of the tissue mass which can be thermally coagulated by one placement of such conventional orthoscopic RF probes 210, 210′. In addition, one or more thermal sensors can be provided in a similar fashion through one or more of the hollow electrode needles 232 to sense temperature, if desired. The lengths of the electrodes and needles 230, 230′, 230″ lie along paths 108 followed by those members when they are extended.

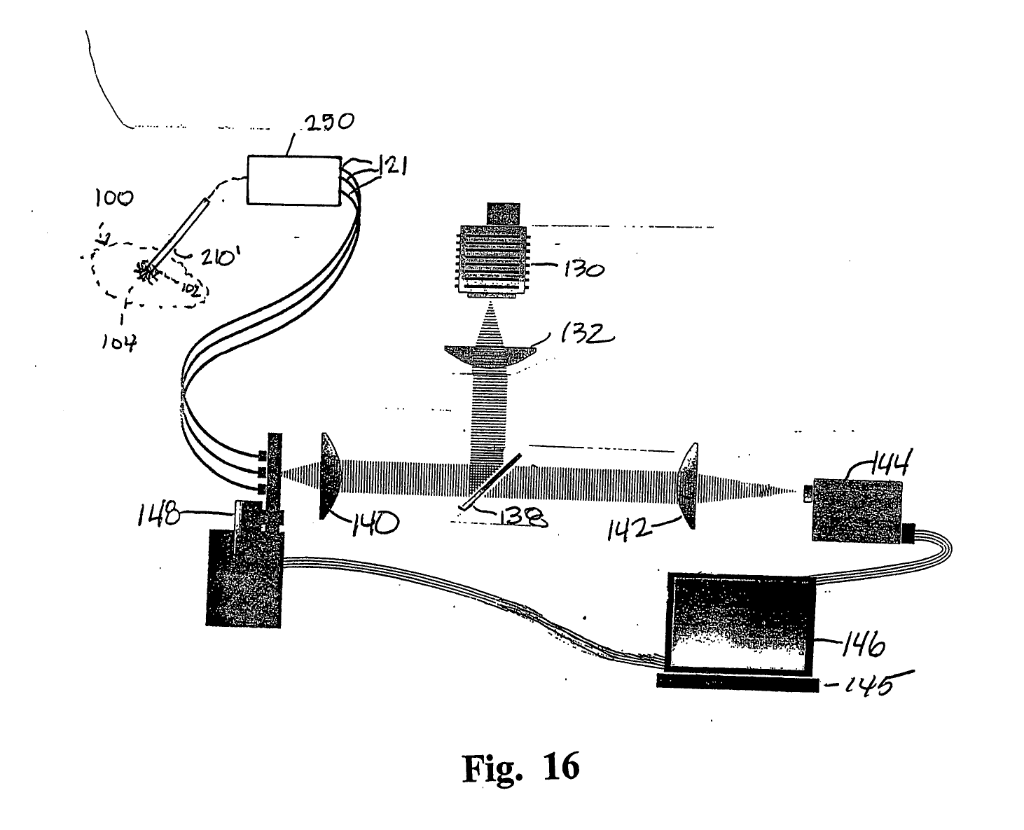

FIG. 16 depicts diagrammatically the remainder of an optical tissue diagnosis system utilizing the probe 210′ of FIG. 14 with needle(s) 230′, 230″ of FIG. 15. This is basically the same as the remainder of the system shown in FIG. 10 with the addition of a device such as a fiber optic coupling wheel 148, to optically couple the various individual optical fibers 121 of the modified needles 230′, 230″ with both the light source(s) 130 and spectrometer 144 and conventional positioning and energy delivery devices and power supplies indicated generally and collectively at 250 for the probe 210′. Further details regarding such systems can be found in U.S. Pat. Nos. 6,622,731, 6,605,085, 6,569,159, 6,551,311, 6,500,175, 6,471,698, 6,330,478, 6,235,023, 6,090,105, 6,080,150, 6,071,280, 6,059,780, 6,053,937, 5,980,517, 5,951,547, 5,935,123, 5,928,229, 5,925,042, 5,913,855, 5,863,290, 5,810,804, 5,800,484, 5,782,827, 5,728,143, 5,672,174, and 5,672,173 all assigned to Rita Medical Systems, Inc., incorporated by reference herein their entirety.

Referring to FIG. 17a, as before, the tumor 102 is generally initially located and the tip of the modified probe 210′ initially positioned in the liver 240 by ultrasound, iMRI, or a frameless surgical navigational system like that disclosed in the aforesaid U.S. Pat. No. 6,584,339 (neither shown). Each modified needle 230′ or 230″ is separately extended to obtain a set of tissue optical spectra to differentiate between the tumorous liver tissue 102 and the normal (non-tumorous) native (uncoagulated) liver tissue 104. If the probe 210′ is off-center, some needles 230′ or 230″ will reach the tumor or tumor boundary more quickly than others. Depending upon the particular situation, the electrodes and/or needles 230, 230′, 230″ may be extended different lengths or the probe 210 moved (as indicated by the large arrow) closer to the center of the tumor 102. If possible, each needle 230′, 230″ is extended sufficiently beyond (or retracted from) the last tumor tissue measurement point to define an acceptable margin around the tumor 102 (or at least around one side of the tumor if the tumor is relatively large, i.e. more than seven centimeters in diameter). At least some (suggestedly at least four) of the normal electrodes 230, which are located as close to the volumetric center of the tumor as possible (based upon the optical data and any other data such as ultrasound, iMRI, etc.), are powered to coagulate the tumor. More preferably, all of the electrodes 230 and the modified needles 230″ can be powered to coagulate the tumor. Thermal coagulation can be monitored with modified needles 230′, 230″, which have been individually positioned beyond the positions of the active needles 230 and the outer surface of the tumor 102 to record spectral changes in the normal or native (i.e. non-tumorous uncoagulated) liver tissue 104 as it is heated or otherwise thermally treated to necrosis. Optical readings (autofluorescence and/or diffuse reflectance) are generated and collected or gathered from the distal end of the needles 230′, 230″ from each monitored optical fiber 120, 120′, 120″ during thermal coagulation. Thermal coagulation is terminated once the appropriate optical spectra signatures of the coagulated normal liver tissue appear at all monitored sites or after a time when certain spectra signature appear.

The utility of the optical criteria identified above, established initially in in vitro canine liver studies, were confirmed in in vivo canine liver studies and have been confirmed as well in in vivo porcine and human liver studies. In particular, several autofluorescent intensity levels were found suitable for identifying both tumor margins and thermal coagulation margins. Porcine livers are widely recognized and used as models for human livers in medical testing.

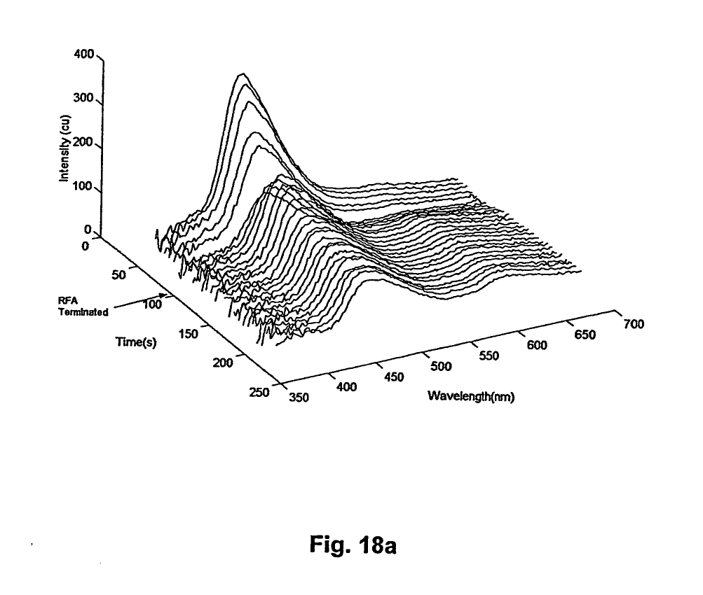

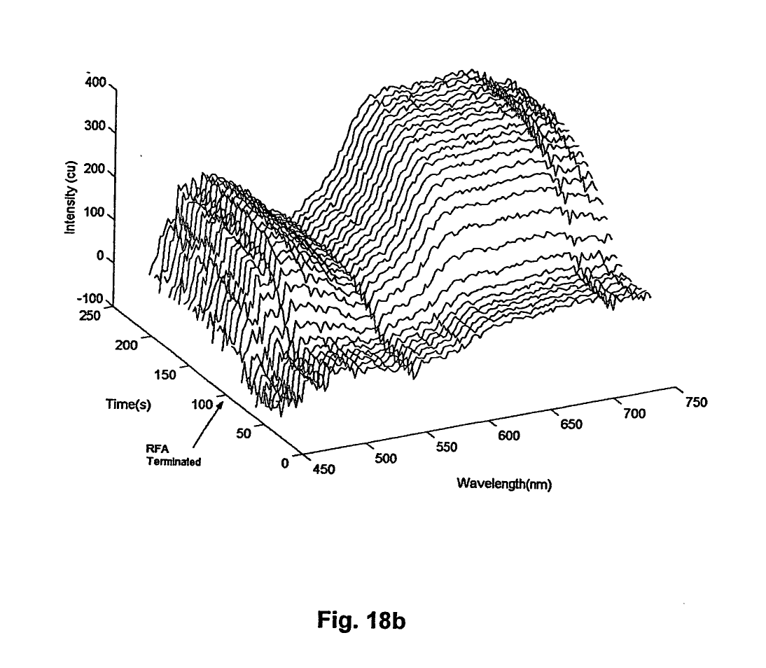

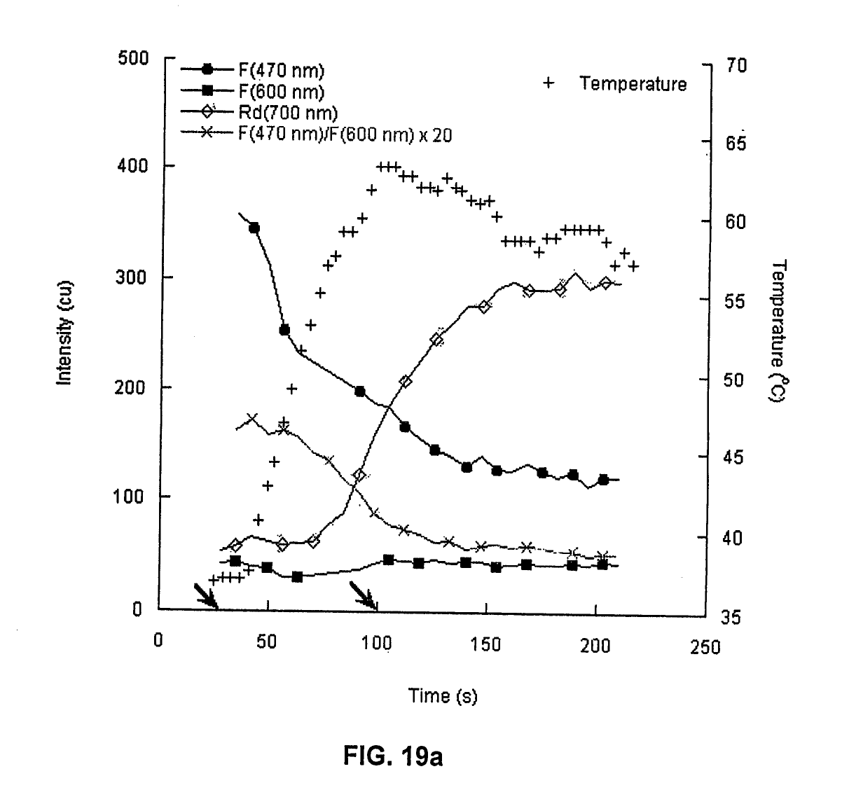

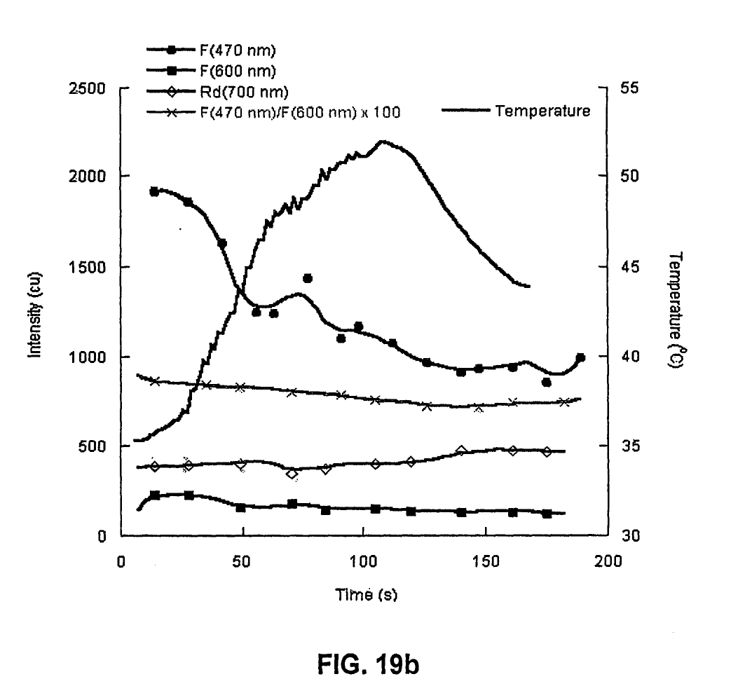

A RF probe like that shown in FIGS. 1 and 2 was tested on five porcine livers in vitro. The time courses of autofluorescence and diffuse reflectance spectra are shown in FIGS. 18a and 18b. Spectral acquisition was performed at a frequency of 0.2 Hz and temperature at a frequency of 1 Hz. Integration time for each spectroscopy characteristic (F and Rd) was one second. Measurements were taken during radio frequency ablation (RFA) in which the RFA generator was operated in a constant power mode at 80 W or lower. FIG. 19a provides a time history of representative spectra values of interest heated to over 60° C. FIG. 19b provides a similar time history of the same spectra but heated to less than 60° C.

The time courses of the porcine liver spectra are essentially the same as those for the canine liver spectra discussed above. Again, the most noticeable changes were in spectra intensity and the changes were wavelength dependent in the same way as were the canine liver spectra changes. Peak fluorescence spectra of the native tissue was at about 470 nm (F₄₇₀) instead of 480 nm and maximum intensity after treatment was at about 500 nm instead of about 510 nm. Diffuse reflectance intensity changes peaked at about 700 nm (Rd₇₀₀) instead of 720 nm. It was found that the decrease trend of fluorescence intensity (F₄₇₀) began almost immediately after local temperatures started to elevate. Diffuse reflectance (Rd₇₀₀), on the other hand, did not exhibit any trends until the local temperature was above about 50° C. Fluorescence intensity at 600 nm (F₆₀₀) remained substantially unaltered during the entire heating-cooling process. The data indicate that fluorescence intensities can be used to monitor initial thermal damage and predict tissue death by heating up to about 50° C. whereas diffuse reflectance is a better indicator of cell destruction above about 50° C.

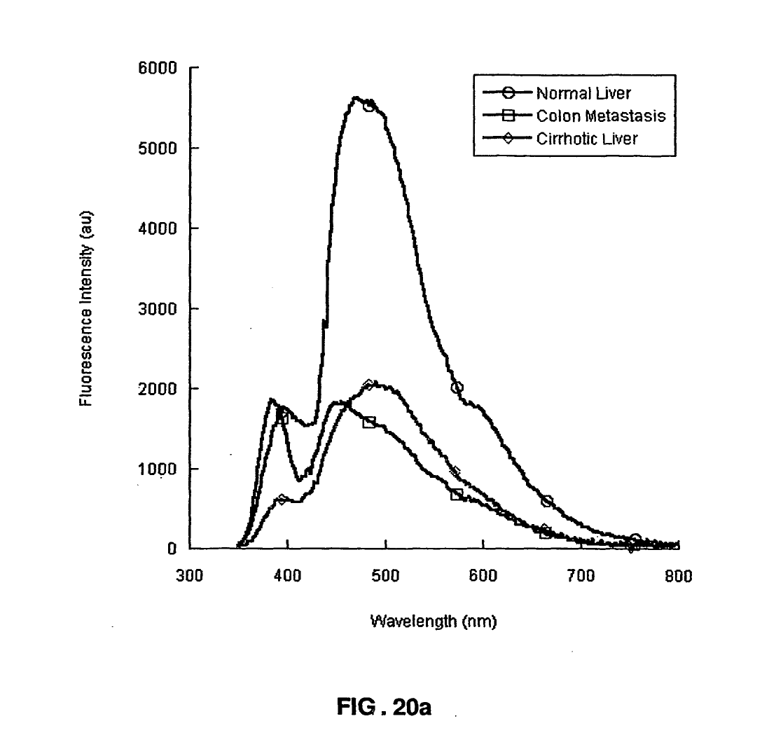

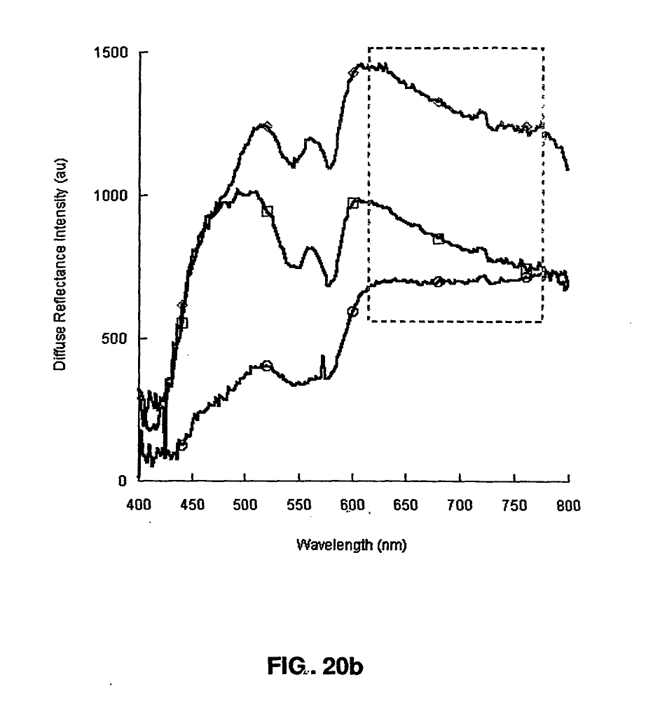

For human liver studies, excitation-emission matrices were generated from in vitro specimens of normal or native liver tissue and from liver tumor (colon metastisis) as well as cirrhotic liver tissues. The specimens where excited with light from 250 nm to 550 nm in length in 10 nm increments and the autofluorescence light emitted analyzed with a spectrometer between 300 nm and 800 nm. Referring to FIG. 20a, all three different types of tissue showed major fluorescence emission peaks at about 280 nm excitation and about 345 nm emission and at about 330 nm excitation and about 380 nm emission. In addition, the cirrhotic tissues and liver tumors examined also possessed a third peak at about 330 nm excitation and about 380 nm emission. This peak was especially pronounced in one tumor sample with necrosis. Therefore fluorescence at about 330 nm is an appropriate optical spectra value to be used to discriminate between normal and tumorous (and cirrhotic) liver tissue.

Next, several other normal, cirrhotic and tumorous tissue samples where tested for fluorescence and diffuse reflectance responses. It was found that fluorescence spectra varied significantly among liver tissue types, as shown in FIGS. 20a, 20b. Referring to FIG. 20a, all the fluorescence spectra from normal liver tissues examined showed one strong peak at about 470 nm emission, the intensity of this primary fluorescence peak varied between 1900 au to 5500 au (2854±548 au, mean±SD, n=7). A secondary peak at about 395 nm emission was also found in the fluorescence spectra from normal liver tissues. In addition, the blood-absorption-induced valleys at about 540 nm and about 580 nm emission are indicated in the fluorescence spectra from the normal liver tissues examined. The fluorescence spectra acquired from cirrhotic liver tissues were found to be relatively similar to those from normal liver tissues; they contained two fluorescence peaks at about 395 nm and about 490 nm emission, respectively. The maximum fluorescence emission intensity from cirrhotic liver tissues (1894±548 au, n=4), however, was smaller than that from normal liver tissues (about 2000 au verses about 5500 au). The FWHM (full width, half maximum) values of the primary fluorescence emission peak from cirrhotic liver tissues (134±4 nm, n=4) were also greater than those from normal liver tissues (121.3±4 nm, n=7). Fluorescence spectra obtained from liver tumors varied greatly in terms of their intensity as well as line-shape. All the fluorescence spectra from the tumorous samples examined possessed a strong peak between about 450 nm and about 500 nm emission. Two of these fluorescence spectra showed three emission peaks at about 380 nm, 430 nm, and 500 nm, with the peak at 380 nm emission being the strongest. The ratios of the peak fluorescence intensities at 380 nm and 480 nm of the liver tumors studied were generally greater than those of the normal liver tissues.

Referring to FIG. 20b, the diffuse reflectance spectra from normal liver tissues were similar in terms of their line-shape. The diffuse reflectance intensity between about 625 nm and about 800 mm remained almost unchanged and the blood absorption signature, signified by the valleys at about 540 nm and about 580 nm emission, was clearly indicated. The diffuse reflectance spectra from liver tumors as well as cirrhotic liver tissues showed an interesting trend above about 600 mm, more particularly, between about 600 nm and about 800 nm emission; the diffuse reflectance intensity monotonically decreased from about 600 nm to about 800 nm. In addition, the diffuse reflectance intensity between about 500 nm and about 600 nm of liver tumors and cirrhotic liver tissues was significantly higher than that of normal liver tissues. These variations indicate that tumorous uncoagulated and non-tumorous uncoagulated liver tissue differentiation can be achieved using fluorescence or diffuse reflectance spectroscopy.

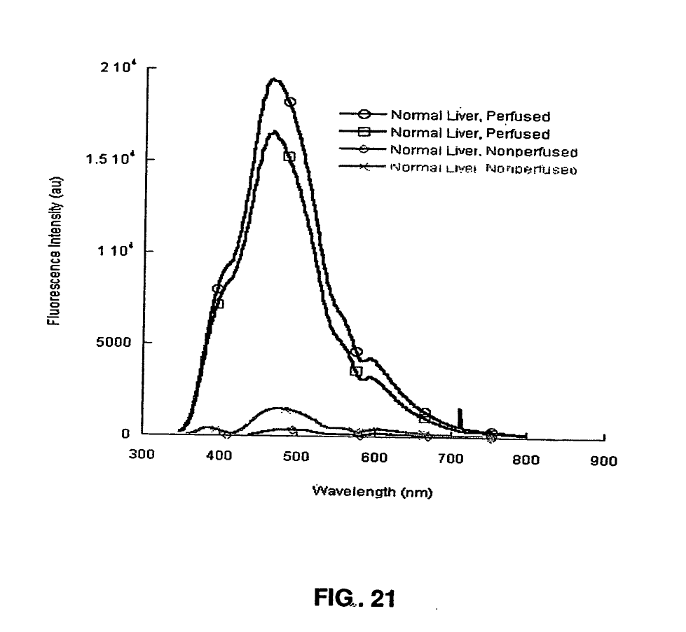

These results were confirmed in vivo. In a clinical study, fluorescence and diffuse reflectance spectra were acquired from perfused (i.e., prior to resection) and non-perfused (i.e., post resection) liver tissue using one of the fiberoptic spectroscopic systems previously described. Two measurements were taken of a sample perfused and nonperfused. As depicted in FIG. 21, fluorescence spectra from perfused liver tissue were much more intense than those from non-perfused tissues. Their line-shapes, however, were similar, with generally the same peaks and valleys.

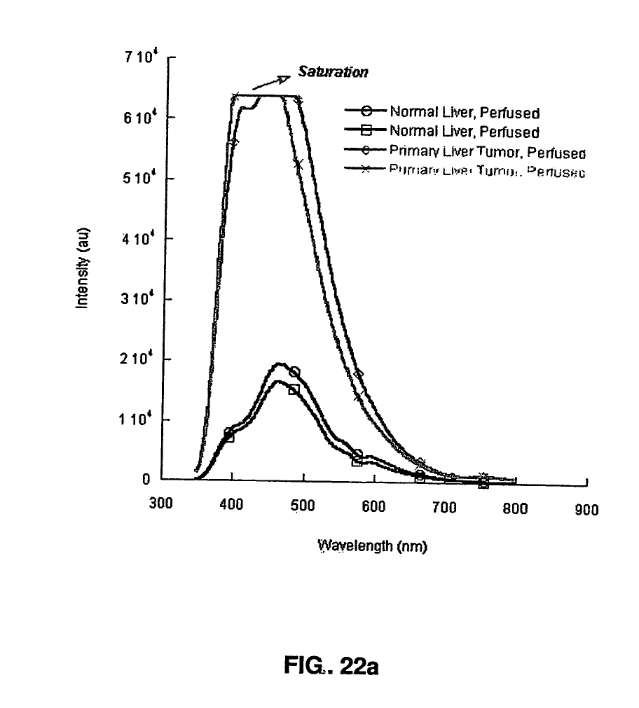

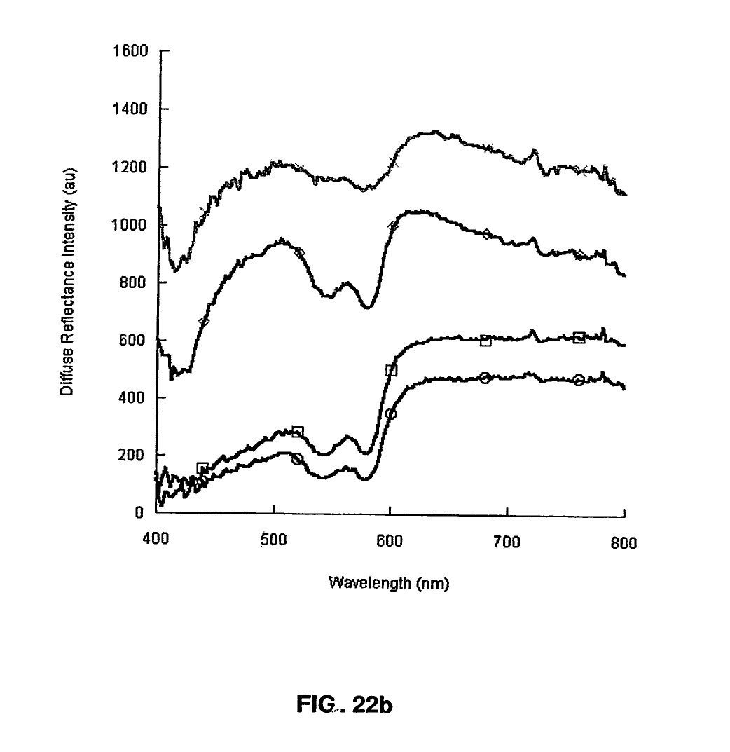

Representative fluorescence and diffuse reflectance spectra from primary liver tumor patients are presented in FIGS. 22a and 22b, respectively. In this case, fluorescence intensities (FIG. 22a) between about 400 nm and about 650 nm emission from primary perfused liver tumors were found to be at least three times greater than those from perfused normal liver tissues. In addition, the blood absorption optical signature, indicated by the valleys at about 540 nm and about 580 nm, was clearly seen in the fluorescence spectra from normal liver tissues but not in those from primary liver tumors. The diffuse reflectance intensities (FIG. 22b) from primary liver tumors were also greater than those from normal liver tissues. Moreover, the diffuse reflectance intensity from primary liver tumors decreased monotonically from about 600 nm to about 700 nm and continued to decrease generally thereafter, while the diffuse reflectance intensity from normal liver tissues remained unchanged in this wavelength region. The results were consistent with the results in the in vitro study described above.

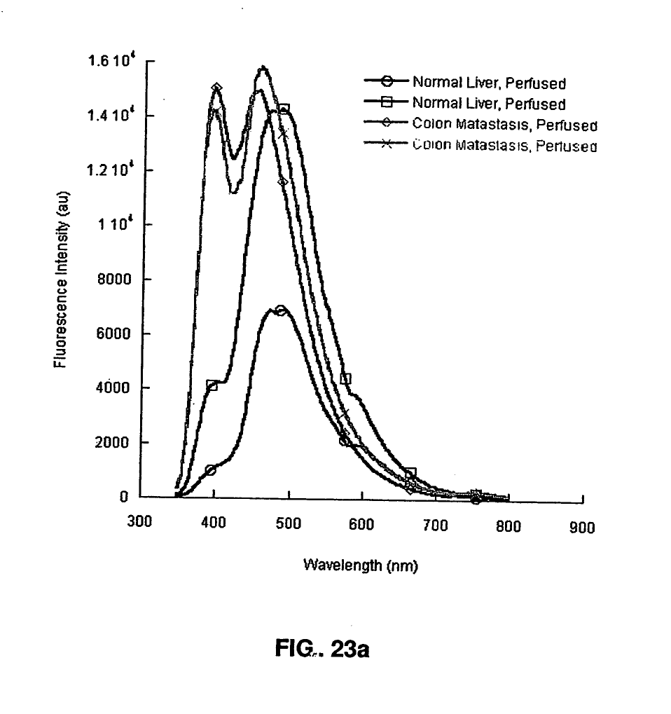

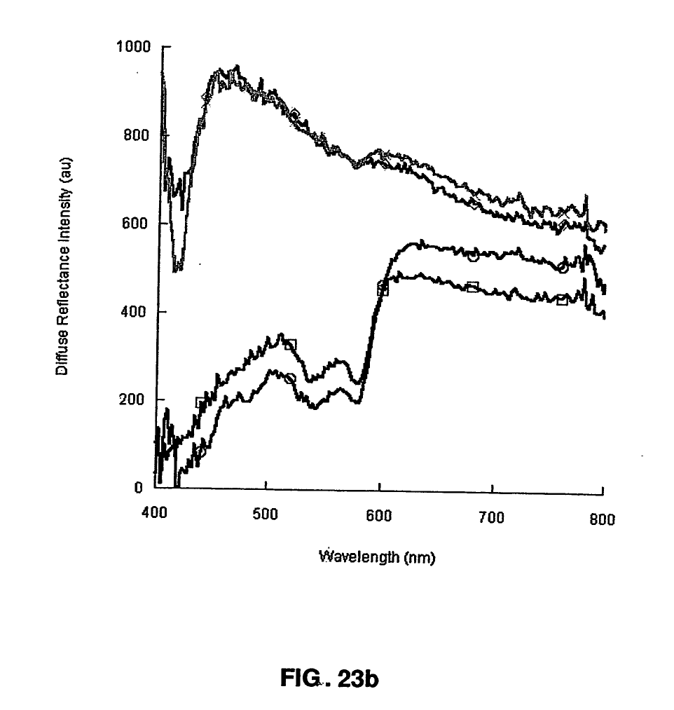

Representative fluorescence and diffuse reflectance spectra from a secondary liver tumor (i.e., colon metastasis) patient are presented in FIGS. 23a and 23b, respectively. The most noticeable difference between the fluorescence spectra from normal liver tissues and those from colon metastasis (FIG. 23a) was the line-shape. The fluorescence spectra from colon metastasis contained two pronounced peaks at about 400 nm and about 480 nm emission, while the fluorescence spectra from normal liver tissues only possessed one broad emission peak with its maximum falling somewhere between about 470 nm and about 500 nm. The diffuse reflectance spectra (FIG. 23b) from colon metastasis were also greater than those from normal liver tissues in terms of the intensity. Moreover, the diffuse reflectance intensity from colon metastasis decreased monotonically from 600 nm to 700 nm emission, while the diffuse reflectance intensity from normal liver tissues remained relatively unchanged in this wavelength region.

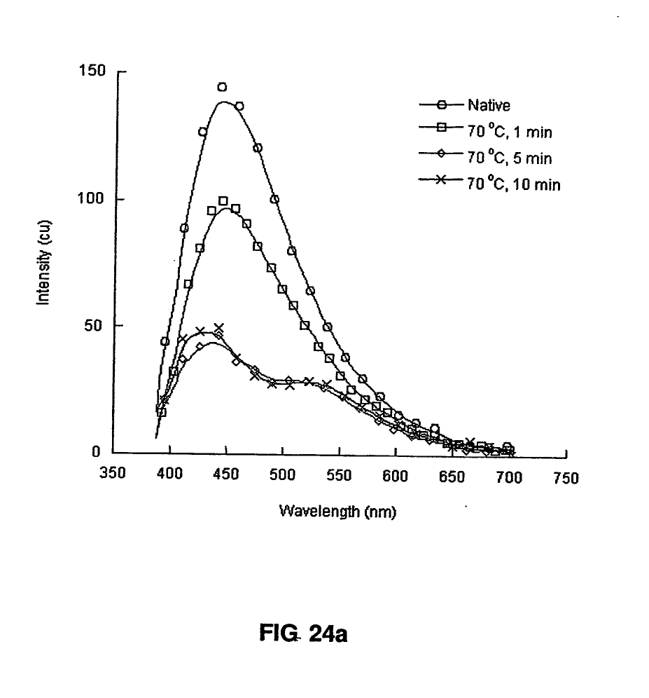

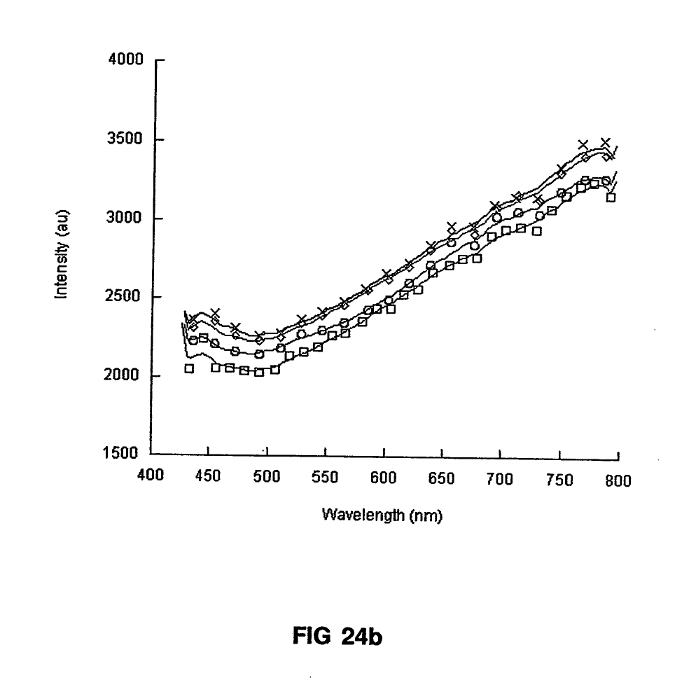

In addition, the effects of heating on hepatocellular carcinoma (HepG2) and human colon adenocarcinoma (SW-480) were investigated. The thermal response of these cells has been found to be similar for the canine, porcine and human livers reported above. Representative autofluorescence (F) spectra and diffuse reluctance (Rd) spectra for active and heavily heated cells are depicted in FIGS. 24a, 24b, respectively. Autofluorescence intensity in FIG. 24a gradually decreased during the course of heating with the most significant decrease found at about 460 nm emission (337 nm excitation) with least pronounce change above about 550 nm. In the HepG2 cells, the fluorescence intensities around 550 nm actually showed a slight increase after about ten minutes of heating in a few occasions. The line shapes of autofluorescence spectra from extensively heated cells (heated five minutes or more) is also significantly different from that of native or lightly heated cells (one minute in FIG. 24a) in that there is one emission peak from unheated cells and two peaks in the two extensively heated cell examples. In contrast, diffuse reflectance spectra from the heated and untreated cells are very similar in terms of their line shape and intensity (FIG. 24b). This would indicate that autofluorescence spectra should be used to monitor and predict denaturation of those cells. SW-480 cells heated at sixty degrees (60° C.) for ten minutes were found to have totally died off within about a forty-eight hour period following a treatment Accordingly, necrosis of these types of tumors by thermal ablation (heating) can be determined by monitoring changes in autofluorescence as well as by temperature or both.

In summation, for native liver tissue differentiation, autofluorescent intensities (F) between about 400 nm and about 600 nm, particularly at or about 400 nm and the ratio of intensities at about 400 nm and about 480 nm (F₄₀₀/F₄₈₀), as well as the full width half maximum autofluorescence intensity of the primary fluorescence emission peak, were found capable of distinguishing between tumorous and non-tumorous liver tissue. Suggestedly, the excitation illumination used to induce autofluorescence is between about 320 nm and about 360 nm. A high pressure 337 nm nitrogen dye laser is a preferred source but light from an appropriate UV light source or filtered light from a broader band light source could also be used. Also, any of these values could be monitored alone or in combination with others of the values. Optical differentiation of margins between tumorous and non-tumorous brain tissue has been discussed in U.S. Pat. No. 6,377,841 B1 and in U.S. Application No. 60/374,707 filed Apr. 22, 2002, both incorporated by reference herein. Similar procedures are followed here and similar equipment can be used for liver tissue discrimination.

For the determination of thermal coagulation, autofluorescent intensities between about 400 nm and about 650 nm, particularly the native tissue peak autofluorescence intensity wavelength between about 460 nm and about 490 nm, the shifted local peak intensity wavelength about 30 nm (±10) greater than the native tissue peak intensity wavelength and the maximum autofluorescence peak intensity wavelength of the denatured/coagulated tissue about 130 nm (±10) above the native tissue peak intensity wavelength, are all spectral values that can be used, preferably together in ratios of the native tissue fluorescence peak with either the locally displaced peak (about +30 nm) or the maximum peak (about +130 nm) of the coagulated tissue. Furthermore, the time derivative of either ratio can be used in particular to diagnose entry into the plateau region of either ratio or to measure a predetermined length of time (e.g. about 30 to about 120 seconds) in the plateau region, to assure coagulation/denaturation. Furthermore, changes in diffuse reflectance intensities particularly in any of the wavelengths in the maximum intensity change range of about 700 nm up to about 750 nm (or their time derivatives) can also be monitored to separate diagnose entry into the plateau region or confirm entry with the autofluorescence values and/or to further monitor heating for a desired predetermined period of time (e.g. about 30 to about 120 seconds) to assure coagulation.

It will be appreciated by those skilled in the art that changes could be made to the embodiments described above without departing from the broad inventive concept thereof. It should further be understood that other spectral correlates are disclosed and suggested to the ordinary practitioner by the above reported data and results. It is understood, therefore, that this invention is not limited to the particular embodiments disclosed, but it is intended to cover all disclosed and suggested variations and modifications within the scope of the present invention as defined by the appended claims.

We claim:

1. A method of identifying internal tissue of an internal organ of a patient comprising the steps of:
inserting a probe into the patient and into an internal area of the organ;
illuminating the internal area of the internal organ against the probe with light carried through the probe;
collecting with the probe light returned from the illuminated tissue;
identifying particular spectral intensity magnitude values using a light detector; and
using one or more of the identified spectral values to identify the illuminated tissue as undenatured non-tumorous, undenatured tumorous or denatured tissue.

2. The method of claim 1 wherein the identifying step comprises converting the collected light with a spectrometer into a plurality of discrete spectral intensity values.

3. The method of claim 1 further comprising of steps of moving the probe incrementally along a path extending at least into the organ; and repeating the illuminating, collecting, identifying and using steps at spaced intervals along the path to identify organ tissue along the path.

4. The method of claim 3 further comprising the step of using the tissue identifications at the spaced intervals to determine a location of a tumor within the organ along the path.

5. The method of claim 4 further of comprising the step of locating a thermal coagulation device in the organ with respect to the tumor using the tumor location determined in the last stated using step.

6. The method of claim 5 further comprising the steps of:
locating an optical sensing end of the probe in non-tumorous tissue adjoining the tumor so as to sense the non-tumorous tissue adjoining the tumor;
thermally coagulating the tumor with the thermal-coagulation device; and
monitoring progress of coagulation of tissue from the tumor into the non-tumorous tissue being sensed by the probe during the thermally coagulating step.

7. The method of claim 1 wherein the steps are performed after thermal coagulation of a tumor within the organ and wherein the inserting step comprises of the step of moving probe along a path through the organ and repeating the illuminating, collecting, identifying and using steps at spaced intervals along the path to identify tissue as coagulated or uncoagulated at the spaced intervals along the path.

8. The method of claim 7 further comprising the step of using the tissue identifications at the spaced intervals to locate a mass of coagulated-tissue within the organ.

9. The method of claim 1 wherein the illuminating step comprises providing through the probe, light from a source with at least an ultraviolet component sufficient to induce autofluorescence in the illuminated tissue.

10. The method of claim 1 wherein the illuminating step comprises providing through the probe, light from a source with a component at least within the range of from about 650 nm to about 750 nm sufficient to illuminate changes in diffuse reflectance occurring between tumorous and non-tumorous organ tissues.

11. A method of diagnosing thermal denaturation of liver tissue comprising the steps of:
locating an optical probe in the tissue in an area immediately adjoining a portion of the tissue to be thermally denaturated;
illuminating tissue immediately adjoining the probe with the probe and collecting with the probe light returned from the illuminated tissue;
converting the collected light with one or more light detectors into a plurality of discrete spectral intensity values; and
using at least a subset of the plurality of discrete spectral intensity values during a thermal denaturation treatment to diagnose denaturation of the illuminated tissue over time.

12. The method of claim 11 wherein the illuminating step comprises providing through the probe, light from a source with at least an ultraviolet component sufficient to induce autofluorescence in the illuminated tissue.

13. The method of claim 12 wherein the collecting step comprises collecting autofluorescence light emitted by the illuminated tissue.

14. The method of claim 11 further comprising before the using step, a preliminary steps of identifying a first wavelength having a maximum intensity value of the plurality; and wherein the using step comprises a step of monitoring a spectral correlate value changing with changes in intensity values of the first wavelength during the thermal denaturation treatment.

15. The method of claim 14 further comprising as part of the preliminary step, pre-selecting a second spectral intensity segment greater in wavelength than the first wavelength and wherein the using step comprising a step of computing ratios of the intensities of the first and second wavelengths over time and using the ratios to diagnose progressive thermal denaturation of the illuminated tissue.

16. The method of claim 15 wherein the preliminary step of pre-selecting the second wavelength further comprises selecting a second wavelength no greater than 50 nm above the first wavelength.

17. The method of claim 15 wherein the step of pre-selecting the second wavelength further comprises selecting a second wavelength greater than 100 nm and no greater than 150 nm above the first wavelength.

18. The method of claim 14 wherein the first wavelength is between 450 and 500 nm.

19. The method of claim 14 wherein the preliminary step comprises identifying a second wavelength of about 30 nm or about 130 nm greater than the first wavelength and wherein the using step comprises monitoring changes in the second wavelength during the denaturation treatment.

20. The method of claim 19 wherein the using step comprises computing ratios of the first wavelength intensities values with the second wavelength intensities values and monitoring changes in the ratios during the denaturation treatment.

21. The method of claim 20 wherein the computed ratios are of the second wavelength intensity values to the first wavelength intensity values and are normalized to an initial ratio value.

22. The method of claim 11 wherein the illuminating step comprises providing to the probe light from a source sufficient to induce diffuse reflectance in a spectral range at least partially overlapping a range between 650 nm and 850 nm.

23. The method of claim 22 wherein the collecting step comprises collecting diffuse reflectance light while the tissue is being illuminate.

24. The method of claim 23 wherein the monitoring step comprises monitoring diffuse reflected intensity values over time during the denaturation treatment for at least one wavelength in a range of 650 nm to 850 nm.

25. A medical tissue ablation system including a tubular member configured for introduction into a patient and having one or more ablation electrodes extending therethrough and individually deployable from a distal open end of the tubular member into an ablation site within the patient, each of the ablation electrodes being coupled with an ablation energy source, characterized by:
a spectrometer; and
at least a first optical fiber encased in a first tubular needle extended through the tubular member with the one or more ablation electrodes and individually extendable from the distal open end of the tubular member into the patient at least proximal to the ablation site, the optical fiber having a first, distal end exposed to light through a first distal open end of the first tubular needle and a second, proximal end optically coupled with the spectrometer so as to deliver to the spectrometer, light collected through the first end of the optical fiber.

26. The system of claim 25 further characterized by: a light source; and at least a second optical fiber having a first distal end extended through the tubular member and from the distal open end of the tubular member into the patient at least proximal to the ablation site and having a second, proximal end optically coupled with the light source.

27. The system of claim 26 further characterized by the light source emitting at least ultra violet light sufficient to induce autofluorescence in tissue illuminated by the second optical fiber.

28. The system of claim 26 further characterized by the light source emitting at least light within a range of between 320 nm and 360 nm sufficient to induce autofluorescence in tissue illuminated by the second optical fiber.

29. The system of claim 28 further characterized by the light source being a laser.

30. The system of claim 26 further characterized by the light source emitting light in a range at least between about 650 nm and about 750 nm to generate diffuses reflectance in tissue illuminated by the light source and the second optical fiber.

31. The system of claim 30 further characterized by the light source being a white light source.

32. The system of claim 31 further characterized by the light source being a halogen lamp.

33. The system of claim 26 further characterized by the second optical fiber being extended through the first tubular needle with the first optical fiber to the first distal open end of the first tubular needle.

34. The system of claim 25 further characterized by the first tubular needle being coupled with the ablation energy source.

35. The system of claim 34 further comprising a thermally insulating cover on the distal end of the tubular needle.

36. The system of claim 34 further comprising a transparent cover over at least the open distal end of the tubular needle.

37. The system of claim 25 further characterized by the second optical fiber having a first distal end being encased in a second hollow needle extended through the tubular member and from the distal open end of the tubular member into the patient at least proximal to the ablation site.

38. The system of claim 37 further characterized by a second distal end of the second tubular needle being coupled with the ablation energy source.

39. The system of claim 38 further comprising a thermally insulating cover on the distal end of the tubular needle.

40. The system of claim 37 further comprising a transparent cover over at least the open distal end of the tubular needle.

41. The system of claim 37 further characterized by the light source being configured to induce autofluorescence in tissue illuminated by the first end of the second optical fiber.

42. The system of claim 37 further characterized by the light source emitting light at least within a range of between 320 nm and 360 nm sufficient to induce autofluorescence in tissue illuminated by the second optical fiber.

43. The system of claim 37 further characterized by the light source being a laser.

44. The system of claim 37 further characterized by the light source having a spectral component at least in the range of 650 nm to 850 nm.

45. The system of claim 37 further characterized by the source being a white light source.

46. The system of claim 37 further characterized by the source being a halogen lamp.
